(19) United States
(12) Patent Application Publication
(10) Pub. No.: US 2019/0042696 A1
(43) Pub. Date: Feb. 7, 2019

(54) Third Generation Sequencing Alignment Algorithm

(71) Applicant: The Board of Trustees of the Leland Stanford Junior University, Stanford, CA (US)

(72) Inventors: Wing H. WONG, Stanford, CA (US); Pegah Tootoonchi AFSHAR, Sunnyvale, CA (US)

(21) Appl. No.: 16/075,885

(22) PCT Filed: Feb. 10, 2017
(86) PCT No.: PCT/US2017/017511

§ 371 Date: Aug. 6, 2018

Related U.S. Application Data
(60) Provisional application No. 62/294,205, filed on Feb. 11, 2016.

(51) Int. Cl. G06F 19/22; C12Q 1/6869; G06F 19/24; G06F 19/26; G06F 19/28

(52) U.S. Cl. CPC G06F 19/22; C12Q 1/6869; G06F 19/28; G06F 19/26; G06F 19/24

(57) ABSTRACT

Methods, software, and systems for aligning a read sequence to a reference sequence are disclosed. In certain embodiments, the methods, software, and systems involve determining similarity of distribution of k-mers between a region of the read sequence and a region of the reference sequence in order to determine whether the region of the read sequence maps to the region of the reference sequence.

### CROSS-REFERENCE

This application claims the benefit of U.S. Provisional Patent Application No. 62/294,205, filed Feb. 11, 2016, which application is incorporated herein by reference in its entirety.

### STATEMENT REGARDING FEDERALLY SPONSORED RESEARCH OR DEVELOPMENT

This invention was made with Government support under contract R01HG007834 awarded by the National Institutes of Health. The Government has certain rights in the invention.

### INTRODUCTION

Whole genome sequencing has revolutionized biology and medicine driving comprehensive characterization of DNA sequence variation, de novo sequencing of a number of species, sequencing of microbiomes, detecting methylated regions of the genome, quantitating transcript abundances, characterizing different isoforms of genes present in a given sample, identifying the degree to which mRNA transcripts are being actively translated, and the like. Indeed the field of pharmacogenomics has expanded exponentially due to the increased availability of genome sequence information of patients.

First and second generation sequencing technologies provide massive throughput at relatively low cost. Third Generation Sequencing (TGS) technologies are the next prominent technique in sequencing based on single-molecule sequencing (SMS). TGS tools generate longer reads compared to First and Second Generation Sequencing Technologies, but they suffer from higher error rates mostly in the form of insertions and deletions (indels).

The process of sequencing DNA includes three basic phases comprising sample preparation, physical sequencing and optionally alignment, and/or re-assembly. Sample preparation involves fragmenting the genome being sequenced and amplification of the fragments. During sequencing the individual bases in each fragment are identified in order, creating individual reads. Bioinformatics software that includes algorithms is then utilized to align overlapping reads, which allows the original genome to be assembled into contiguous sequences.

Currently, commonly used algorithms for aligning individual long reads to a reference sequence or dataset, are based on modified versions of the seed-and-extension concept. Such methods often start by finding exact matches between query and reference sequence, then greedily finding optimal seed chains and extending them using dynamic programming with optional drop-off heuristics to avoid extension over poor regions.

The methods, software, and systems provided in the present disclosure provide a robust approach to locate the sequencing position of a read enabling alignment and assembly of sequence reads that may include aberrations such as insertions and/or deletions.

### SUMMARY

The present disclosure provides methods, systems, executable software products, and storage devices for aligning a read sequence to a reference sequence. In certain embodiments, a method for aligning a read sequence to a reference sequence segment is disclosed. The method may include creating a window for the read sequence and a window for the reference sequence segment, wherein the windows are of the same length; computing the numbers of occurrences of unique k-mers within each window, computing a k-mer count similarity value based on the numbers of occurrences of the unique k-mers within each window; performing steps (a)-(c) iteratively for a plurality of windows across the read sequence and a plurality of windows across the reference sequence segment, thereby computing a plurality of k-mer count similarity values, wherein the beginning of each subsequent window in each of the read sequence and of the reference sequence segment is offset from the beginning of the previous window in the respective sequence by a distance d; calculating a similarity score by averaging the plurality of k-mer count similarity values; and aligning the read sequence to the reference sequence segment when the similarity score is above a threshold, wherein the windows created in the first performance of step (a) are positioned at the start of each sequence.

In certain embodiments, the method may include repeating steps (a)-(f) are for the read sequence and a different segment of the reference sequence.

In certain embodiments, reference sequence segment may be a region of a reference sequence obtained from a genome database. In certain embodiments, the reference sequence may be a read sequence. In certain embodiments, the reference sequence may be a read sequence obtained from sequencing the same sample from which the sequence of the read sequence is obtained.

In certain embodiments, the length of each of the windows may be at least 50 bases. In certain embodiments, the length of each of the windows may be any whole number value ranging from 1-10,000 bases, wherein the length is held constant.

In certain embodiments, the distance d may be at least 10 bases long. In certain embodiments, the distance d may range from 1-500 bases in length, wherein d is held constant.

In certain embodiments, the k-mer may be 2-10 bases in length. In certain embodiments, the k-mer may be 3 bases in length. In certain embodiments, the k-mer may be 4 bases in length.

Also disclosed herein is an executable software product stored on a computer-readable medium. In certain embodiments, the executable software product stored on a computer-readable medium may contain program instructions for the conducting the above disclosed methods.

A system configured to execute instructions to conduct the above disclosed methods is also provided. The system may include a memory with stored instructions to carry out the above disclosed methods and a processor coupled to the memory and configured to execute instructions in the memory.

In certain embodiments, a storage device storing instructions executable for performing the above disclosed methods are disclosed.

### BRIEF DESCRIPTION OF THE FIGURES

FIG. 1 depicts a reference sequence segment of a reference sequence and exemplary windows for reference sequence segment and for a read sequence.

FIG. 2 depicts an embodiment for counting k-mers within a window of a reference sequence segment and within a corresponding window of a read sequence.

FIG. 3 depicts a plurality of windows in a reference sequence segment and a read sequence.

FIG. 4 depicts a schematic for the comparison of the read sequence to a plurality of segments of the reference sequence.

FIG. 5 depicts the computed similarity scores for alignments of the read sequence across the reference sequence.

FIG. 6 illustrates one embodiment of a computer for carrying out the disclosed methods.

FIG. 7 depicts the distribution of cosine distance between random positions in the E. coli genome with its mutated (substitutions only) version using k=3.

FIG. 8 is a continuation of FIG. 7.

FIG. 9 depicts the distribution of cosine distance between random positions in the E. coli genome and with its mutated version (substitutions only) using k=4.

FIG. 10 is a continuation of FIG. 9.

FIG. 11 depicts a distribution of cosine distance between 1000 random sequences of length 5000 bases from the E. coli genome and with respect to their mutated versions (substitutions and indels), using k=3.

FIG. 12 depicts a distribution of cosine distance between 1000 random sequences of length 5000 bps from the E. coli genome and with respect to their mutated versions (substitutions and indels), using k=4.

FIG. 13 depicts the cosine similarity scores for a read sequence of length 5000 bases from the E. coli genome compared across the entire genome with simulated error rates of 15% and 35% with k=3 and d=10.

FIG. 14 centers and zooms around the expected alignment position (vertical dotted line) from FIG. 13, depicting the cosine similarity score for a read sequence of length 5000 bases from the E. coli genome compared with the E. coli genome around the sampled position with simulated error rates of 15% and 35%, with k=3 and d=10.

### DEFINITIONS

All publications, patents and patent applications cited herein, whether supra or infra, are hereby incorporated by reference in their entireties.

In describing the present invention, the following terms will be employed, and are intended to be defined as indicated below.

It must be noted that, as used in this specification and the appended claims, the singular forms “a”, “an” and “the” include plural referents unless the content clearly dictates otherwise. It is further noted that the claims can be drafted to exclude any optional element. As such, this statement is intended to serve as antecedent basis for use of such exclusive terminology as “solely,” “only” and the like in connection with the recitation of claim elements, or use of a “negative” limitation.

As used herein, the term “aligning” or grammatical equivalent thereof refers to a mapping a read sequence to a region in a reference sequence.

As used herein, the term “read sequence” refers to a sequence of contiguous nucleotides determined from a single segment of a sample nucleic acid by a sequencing instrument. A single segment may be an amplification product generated by amplification of the genome or a portion of the genome being sequenced. The sequence of contiguous nucleotides from a single segment of the sample nucleic acid may be represented as a stream of data generated by a sequencing technique, which data is generated, for example, by means of base-calling software associated with the sequencing technique. e.g., base-calling software from a commercial provider of a DNA sequencing platform. A read sequence may also be referred to as a “query sequence” or a “sequence read”.

As used herein, the term “reference sequence” refers to a known sequence of contiguous nucleotides of the genome or a portion of the genome of an organism. A reference sequence may be used as the input sequence to which a read sequence is aligned. The reference sequence to be used depends on the origin of the read sequence. The reference sequence may be a sequence of nucleic acid from the same species as the species from which the read sequence is obtained. If the sequence from the same species is not available, then the sequence of an organism most closely related to the organism whose genome is being sequenced may be used as the reference sequence. The reference sequence may be determined by a sequencing technique or may be obtained from a sequence database, such as an organism's genome obtained from the genome library of the National Center for Biotechnology Information. The reference sequence may also be a read sequence. Aligning a read sequence to a read sequence, where the read sequences are obtained from sequencing a nucleic acid sample, is useful for finding regions of overlap in the read sequences and assembly of the read sequences to yield a longer contiguous read sequence.

As used herein, the term “data structure” refers an organization of information, usually in a computer or memory device. Data structure allows for efficient execution of algorithm that processes the information/data. Exemplary data structures include dictionary, queues, stacks, linked lists, heaps, hash tables, arrays, trees, and the like. Data structures may have substructures that correspond to units of information or to subsets of related information. For example, arrays have rows and columns of entries; trees have nodes, branches, subtrees, and leaves; or the like. An exemplary data structure may include a list of all possible unique k-mers and a count indicator for the number of occurrences of a unique k-mer of the list in a read and a reference sequence.

As used herein, the term “identity” in the context of two sequences refers to an exact nucleotide-to-nucleotide or amino acid-to-amino acid correspondence of two polynucleotides or polypeptide sequences, respectively. Percent identity can be determined by a direct comparison of the sequence information between two molecules by aligning the sequences, counting the exact number of matches between the two aligned sequences, dividing by the length of the shorter sequence, and multiplying the result by 100. Readily available computer programs can be used to aid in the analysis, such as ALIGN, Dayhoff, M. O. in Atlas of Protein Sequence and Structure M. O. Dayhoff ed., 5 Suppl. 3:353-358, National biomedical Research Foundation, Washington, D.C., which adapts the local homology algorithm of Smith and Waterman Advances in Appl. Math. 2:482-489, 1981 for peptide analysis. Programs for determining nucleotide sequence identity are available in the Wisconsin Sequence Analysis Package, Version 8 (available from Genetics Computer Group, Madison, Wis.) for example, the BESTFIT, FASTA and GAP programs, which also rely on the Smith and Waterman algorithm. These programs are readily utilized with the default parameters recommended by the manufacturer and described in the Wisconsin Sequence Analysis Package referred to above. For example, percent identity of a particular nucleotide sequence to a reference sequence can be determined using the homology algorithm of Smith and Waterman with a default scoring table and a gap penalty of six nucleotide positions.

The terms “polynucleotide,” “nucleic acid” and “nucleic acid molecule” are used herein to include a polymeric form of nucleotides of any length, either ribonucleotides or deoxyribonucleotides. This term refers only to the primary structure of the molecule. Thus, the term includes triple-, double- and single-stranded DNA, as well as triple-, double- and single-stranded RNA. It also includes modifications, such as by methylation and/or by capping, and unmodified forms of the polynucleotide. More particularly, the terms “polynucleotide,” “nucleic acid” and “nucleic acid molecule” include polydeoxyribonucleotides (containing 2-deoxy-D-ribose), polyribonucleotides (containing D-ribose), any other type of polynucleotide which is an N- or C-glycoside of a purine or pyrimidine base, and other polymers containing nonnucleotidic backbones.

“Target nucleic acid” or “target nucleotide sequence,” as used herein, refers to any nucleic acid that is of interest for which the nucleotide sequence is to be determined.

### DETAILED DESCRIPTION

The present disclosure provides methods, software and systems for aligning a read sequence to a reference sequence.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments are specifically embraced by the present invention and are disclosed herein just as if each and every combination was individually and explicitly disclosed, to the extent that such combinations embrace operable processes and/or devices/systems. In addition, all sub-combinations listed in the embodiments describing such variables are also specifically embraced by the present invention and are disclosed herein just as if each and every such sub-combination of chemical groups was individually and explicitly disclosed herein.

### Methods

The present disclosure provides methods for aligning a read sequence to a region of a reference sequence. The read sequence is also referred to as a query sequence. The alignment methods may involve (a) creating a window for the read sequence and a window for a segment of the reference sequence, which windows are of the same length; (b) computing the numbers of occurrences of unique k-mers within each window, wherein the k-mers are of the same length; (c) computing a k-mer count similarity value based on the numbers of occurrences of the unique k-mers within each window; (d) performing steps (a)-(c) iteratively for a plurality of windows across the read sequence and a plurality of windows across the segment of the reference sequence, where the beginning of a subsequent window in each of the read sequence and of the segment of the reference sequence is offset from the beginning of the previous window in the respective sequences by a distance d, where d is the same between corresponding windows in the read sequence and the reference sequence; (e) calculating a similarity score by averaging the computed k-mer count similarity values; and (f) aligning the read sequence to the segment of the reference sequence if the similarity score is above a threshold, where the windows created in step (a) are positioned at the start of the read sequence and the segment of the reference sequence.

In certain embodiments, the step (a) of creating a window may involve positioning a window which starts at the first nucleotide of each of the read sequence and the segment of the reference sequence. In certain embodiments, the step of creating additional windows downstream of the initial windows may involve selecting a region or subsequence in the read sequence and the reference sequence segment at which the additional windows are positioned. For example, the additional windows in each of the read sequence and the segment of the reference sequence may be offset from the window immediately upstream from it by a distance d which may be about 1 or more bases. The offset distance d may be held constant for each of the windows. In other words, the windows in each of the read sequence and the segment of the reference sequence is offset from the previous window by the same distance.

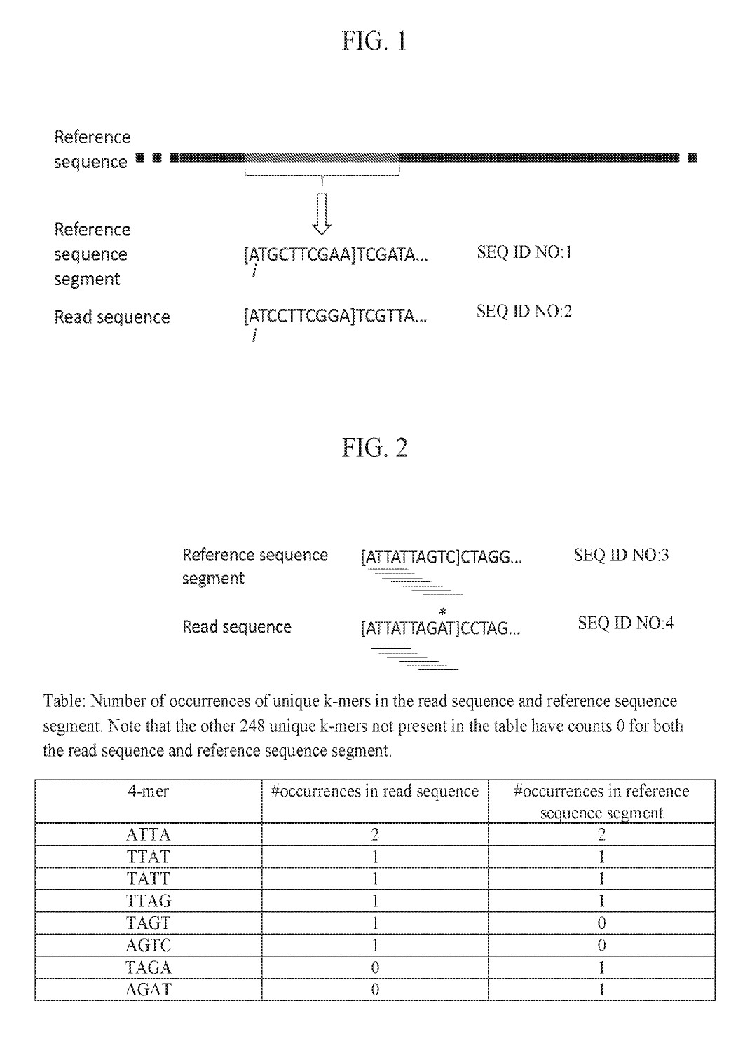

The length/size of the window can be denoted by w which may range from 1-10,000 bases, for example, 100-10,000 bases, 10-5000 bases, 50-1000 bases, 100-1000 bases, 100-800 bases, 100-700 bases, 50-1000 bases, 50-800 bases, 50-700 bases, 50-500 bases, 100-500 bases, 300-700 bases, 400-700 bases, or 400-600 bases. In certain embodiments, the window size w is constant for a single alignment between read sequence and reference sequence segment. In other words, all windows created for a single alignment may have the same length. In some instances, the read sequence and reference sequence segment may be similar in length. In other instances, the read sequence and reference sequence segment may have the same length. The window may be used to denote a region of a sequence [i, i+w], where i is an index whole number denoting the position in the read sequence or reference sequence segment and w is the window length. FIG. 1 illustrates an example showing a schematic of a reference sequence in which a segment is selected for comparison to a read sequence. A segment (grey region) of the reference sequence is depicted in FIG. 1. FIG. 1 also shows a window (denoted by square brackets) of length 10 bases starting at position i. A corresponding window of length 10 bases starting at position i is created similarly for a read sequence. In this example, the read sequence and reference sequence segment are not identical. It is noted that a window size of 10 bases is for illustration purposes only. As noted herein, the length w of the subsequent windows positioned downstream of the depicted windows is held constant.

In certain embodiments, the numbers of occurrences of each possible unique k-mer (also referred to as a k-mer distribution or k-mer count distribution), within each window may be computed by counting and keeping track of each instance of every possible unique k-mer. In certain embodiments, the nucleotide sequence in a window in the read sequence may be used to generate a list of all overlapping k-mers, and the nucleotide sequence in the corresponding window (starting at the same position i) in the segment of the reference sequence may also be used to generate a list of all overlapping k-mers. The number of unique k-mers may be counted for each window to determine the similarity in the number of occurrences of unique k-mers in each window. In other embodiments, a data structure may be used for counting the unique k-mers. The higher the similarity in the number of occurrences of each unique k-mer in a pair of windows, the higher the k-mer count similarity value.

k-mers may be 3, 4, 5, 6, 7, 8, 9, 10, 20, 30, 40, 50, 60, 70, 80, 90 or 100 bases in length; for example, k-mers may range from 3-100, 3-80, 3-50, 3-80, 3-10, 3-5, 4-100, 4-80, 4-50, 4-80, 4-10, 4-5, 2-4, 2-10, or 3-4 bases in length. As noted herein, within a window the size of the k-mers is held constant. In certain embodiments, k-mer size is held constant in all of windows generated for the alignment method. Within each window, consecutive k-mers may overlap by at least 1 base, at least 2 bases, at least 3, or at most k−1 bases (e.g., for a 10 nucleotides long k-mer, consecutive k-mers may overlap by at most 9 bases). In certain embodiments, the overlap between consecutive k-mers across a window in the read sequence and across the corresponding window in the segment of the reference sequence is constant. In certain embodiments, the overlap length between adjacent k-mers is constant for the entire alignment method. For example, consecutive 3-mers may overlap by 1 or 2 bases and consecutive 4-mers may overlap by 1, 2, or 3 bases. In certain embodiments, consecutive 3-mers may overlap by 2 bases and consecutive 4-mers may overlap by 3 bases. In certain embodiments, consecutive k-mers may not overlap. For example, consecutive k-mers may be separated by 1-3000 nucleotides, such as, 50-1000 bases, 100-1000 bases, 100-800 bases, 100-700 bases, 50-1000 bases, 50-800 bases, 50-700 bases, 50-500 bases, 100-500 bases, 300-700 bases, 400-700 bases, or 400-600 bases. In certain embodiments, k-mer size may be constant across the entire window and k-mers across the entire window may be counted. For instance, as shown in FIG. 2, for a window length of 10 bases, counting all of the 4-mers overlapping with the previous k-mer by 3 bases, seven 4-mers would be counted across the entire length of the window for the read sequence and for the segment of the reference sequence.

FIG. 2 shows a first window of length 10 (shown with square brackets) of a read sequence with 4-mers overlapping by 3 bases for a read sequence and a reference sequence segment, where the read sequence has an insertion of the nucleotide “A” at position 9 (indicated by a *), relative to the reference sequence segment. All of the counted 4-mers are underlined and the counts of unique k-mers within the windows are tabulated in the table shown in FIG. 2. Note that when k=4, as in this example, there are 4⁴, or 256 unique k-mers. Thus, for the 248 other unique k-mers not presented in the table in FIG. 2, the read sequence and reference sequence segment will have 0 counts.

In the presence of insertions and deletions, aligned sequences have their error-free segments shifted with offset o, where o=lins−dell. Whereas frameshifts caused by such insertions and deletions are often detrimental to the accuracy of alignment techniques using direct cross-correlation for longer read sequences, counting k-mers circumnavigates this problem by analyzing the content of short k-mers as a measure of local similarity. Take for example the read sequence and reference sequence fragment below,

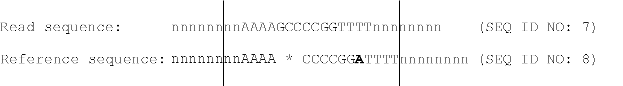

where n is any nucleotide, * is a deletion, bold letters are insertions, and the vertical lines define the boundaries of the window (length 17). A method simply searching for corresponding identical, consecutive nucleotides in the two sequences would find CCCCGG at best. However, the method disclosed herein determines the number of occurrences of k-mers, i.e., a distribution for the k-mers, resulting in a larger local alignment.

The k-mers in the designated window of the read sequence are as follows: XAAA, AAAA, AAAG, AAGC, AGCC, GCCC, CCCC, CCCG, CCGG, CGGT, GGTT, GTTT, TTTT, TTTX. The k-mers in the corresponding or parallel window of the reference sequence are as follows: XAAA, AAAA, AAAC, AACC, ACCC, CCCC, CCCG, CCGG, CGGA, GGAT, GATT, ATTT, TTTT.

The underlined k-mers appear in both the read sequence and reference sequence once in this example. This example illustrates how identical segments are identified across the entire window and used to map a read sequence to a region of the reference sequence even when the read sequence is not identical to the reference sequence.

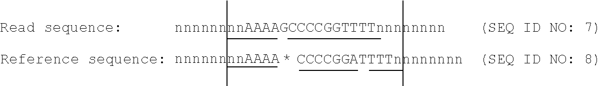

As the window sizes approach larger numbers, such as 500, the method disclosed herein becomes more forgiving with respect to indels, as correlating k-mers may still lie within corresponding windows, despite insertions and deletions increasing the offset between them.

This method of identifying the numbers of occurrences of k-mers, or k-mer counts, allows for the alignment method to be more robust in the face of indels than other cross-correlation alignment methods.

In some instances, the number of occurrences of unique k-mers may be counted by creating k-mer count vectors Vᵢ¹ and Vᵢ² for the read sequence and the reference sequence segment, respectively, where i is the position in the sequence where the window starts, and

Vᵢ=[n₀,n₁, . . . ,n_{q}], where 0≤i<l−w+1, [1]

where nⱼ=count(uⱼ) in the window,

where Q=list of k-mers {vⱼ}, for 0≤j<q, and

q=|Σ|^{k}, where Σ={A, T, G, C}.

For example, when counting the numbers of occurrences of all unique 3-mers for a DNA sequence, then k=3, q=64 total possible unique 3-mers.

In certain embodiments, a k-mer that includes an unknown base(s) may be randomly mapped to one of q possibilities, thereby keeping the possible unique k-mer pool restricted to combinations of Σ. In certain embodiments where there is a k-mer that includes an unknown base(s), q+1 possibilities for vⱼ may be considered, where q contains the k-mer with unknown base(s) and that unique k-mer is counted for both the read sequence and reference sequence. Doing so would be analogous to introducing another dimension in k-mer space for set of unknown k-mer(s).

In some embodiments, the k-mer count similarity value may be computed based on the numbers of occurrences of the unique k-mers within the corresponding windows of the read sequence and the reference sequence segment. The k-mer count similarity value, which may also be referred to as a k-mer distribution similarity value or k-mer count distribution similarity value, may be calculated by using the following cosine similarity formula between the k-mer count vectors of the read sequence and reference sequence segment:

$${S}_{i} = \frac{{V}_{i}^{1} \cdot {V}_{i}^{2}}{‖ {V}_{i}^{1} ‖ ‖ {V}_{i}^{2} ‖} \qquad [ 2 ]$$

This k-mer count similarity value or score (Sᵢ) represents the local similarity of the sequence fragments f¹ and f² at locally aligned positions in the read sequence and reference sequence segments, respectively, where fragments f¹ is defined by a window and f² is defined by the corresponding window. Using cosine similarity score compared to other metrics provides the advantage that a global similarity score (Eq. 4) can be implemented efficiently using Fast Fourier Transom (FFT). In some embodiments, other similarity metrics may be used, such as Euclidean distance.

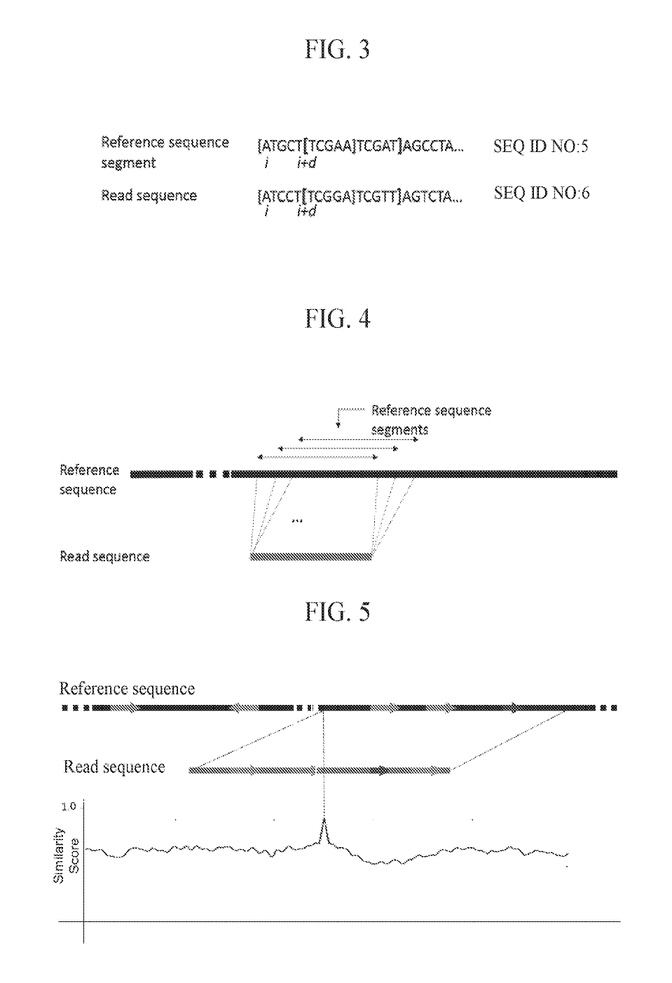

In certain embodiments, the steps of creating windows, counting unique k-mers, and computing a k-mer count similarity value iteratively for a plurality of windows across the read sequence and reference sequence segment provide a plurality of k-mer count similarity values. The steps of creating windows, counting unique k-mers, and computing a k-mer count similarity value may be performed till the entire length of the read sequence has been compared to the segment of the reference sequence. In certain embodiments, the steps of creating windows, counting unique k-mers, and computing a k-mer count similarity value may be carried out till the entire length of the read sequence has been compared to another read sequences. In certain embodiments, the steps of creating windows, counting unique k-mers, and computing a k-mer count similarity value may be carried out until at least a 500 nucleotide long stretch of the read sequence has been compared to a reference sequence. As such, in certain embodiments, k-mer count similarity values may be computed for at least a 500 base long stretch of the read sequence, for example, 700 bases, 1000 bases, 3000 bases, 5000 bases, 7000 bases, 10,000 bases, 13,000 bases, 15,000 bases, 18,000 bases, 20,000 bases, or up to 50,000 bases. While the steps for computing k-mer similarity values are performed iteratively, it is not necessary to compute the k-mer similarity values for different pairs of windows (corresponding windows in the read and reference sequences) sequentially. In certain embodiments, the steps (a)-(c) may be performed simultaneously for a plurality of pairs of windows. As noted herein, each subsequent window in each of the read sequence and the reference sequence segment is offset from the beginning of the previous window in the respective sequence by a distance d. This window offset distance d can vary in size and can have sizes ranging from 1-1000 bases, 1-500 bases, 1-100 bases, 5-100 bases, 10-100 bases, 50-100 bases, 1-20 bases, or 1-10 bases, 10-50 bases in length. In certain embodiments, the offset distance d between windows is constant for a whole alignment. In certain cases, adjacent windows may be overlapping. In certain cases, adjacent windows may not overlap. In certain cases, adjacent windows that do not overlap may be immediately adjacent to each other, i.e., not separated by intervening nucleotides. In certain embodiments, the window size may be 500 bases and the offset d may be 10 bases. In other embodiments, different combinations of window sizes and offsets are possible, such as a window size of 50 bases with an offset of 5 bases, a window size of 100 bases with an offset of 10 bases, a window size of 500 bases with an offset of 50 bases, a window size of 1000 bases with an offset of 100 bases, or a window size of 5000 bases with an offset of 100 bases. FIG. 3 shows a subsequent window (bold square brackets) offset from the previous window starting at position i (brackets) by distance d=5, where w=10 for both the read sequence and the reference sequence segment. In this example, the read sequence and reference sequence segment are not identical. It is noted that window size of 10 bases and offset of 5 is for illustration purposes only.

As noted herein, d is the same or equal between corresponding windows in the read sequence and reference sequence segment. For instance, a second window created for each of the read sequence and reference sequence segment may be offset from the first window in each of the respective sequences by 10 bases. As explained, windows are created iteratively and a k-mer count similarity value is computed for each window, using the aforementioned methods. In certain embodiments, an overall similarity score between the read sequence and reference sequence segment is computed by averaging of the plurality of k-mer count similarity values which may be calculated using the formula [2] to calculate similarity scores (Sᵢ). The overall similarity score may be calculated by the following equation:

$$S = \frac{1}{\vert γ \vert} {Σ}_{( i ) ∈ γ} {S}_{i} \\ where \\ γ = \{ ( {i}^{1} , {i}^{2} ) , ( {i}^{1} + d , {i}^{2} + d ) , … , ( {i}^{1} + pd , {i}^{2} + pd ) \} \\ p < ⌊ \frac{min ( {l}_{read} , {l}_{reference} )}{d} ⌋ , \qquad [ 3 ]$$

and i¹ and i² represent the start index of the read sequence and reference sequence segment, respectively. Computing Equation 3 across a reference sequence using cosine similarity as metric can be formulated as cross correlation and can be computed efficiently using FFT.

In some instances, the calculation of similarity score may be repeated iteratively for the read sequence and a different segment, or region, of the reference sequence. FIG. 4 shows how the read sequence may be compared to a plurality of segments in the reference sequence. In certain aspects where a read sequence is compared to a plurality of segments in the reference sequence, the read sequence may be compared, and subsequently a similarity score computed, for every positioning of the read sequence along the reference sequence; in other words, comparing the read sequence to the reference sequence starting at every sampled position i of the reference sequence, where 0≤i<l_{reference}−l_{read}+1. In such embodiments, the read sequence and reference sequence may be considered to be compared at an aligned offset m from the start of the reference sequence. In such embodiments, the cosine similarity score may be calculated by

$${S}_{{i}^{1} {i}^{2}} = \frac{{V}_{{i}^{1}}^{1} \cdot {V}_{{i}^{2}}^{2}}{‖ {V}_{{i}^{2}}^{1} ‖ ‖ {V}_{{i}^{2}}^{2} ‖} ,$$

where m=i²−i¹ and i₀¹ and i₀² are the start positions of aligned first windows of length w in read and reference sequence, respectively. The overall similarity score between two sequences aligned with offset m calculated by

$$S [ m ] = \frac{1}{\vert γ \vert} {Σ}_{( {i}^{1} {i}^{2} ) ∈ {γ}_{m}} {S}_{{i}^{1} {i}^{2}} \\ where \\ {γ}_{m} = \{ ( {i}_{0}^{1} , {i}_{0}^{2} ) , ( {i}_{0}^{1} + d , {i}_{0}^{2} + d ) , … , ( {i}_{0}^{1} + pd , {i}_{0}^{2} + pd ) \} \\ p < ⌊ \frac{min ( {l}_{read} , {l}_{reference} )}{d} ⌋ , \\ {i}_{0}^{2} - {i}_{0}^{1} = m . \qquad [ 4 ]$$

and for the purpose of global alignment, i₀¹ might be set to 0 and i₀²=m.
Eq. 4 might be computed efficiently to find overall similarity score (global alignment) of a read sequence against reference sequence using Fast Fourier Transform (FFT) for

$$m = 0 , d , … , ⌊ \frac{{l}_{reference}}{d} ⌋ d .$$

For a sequence of length l, Vᵢ is defined as in Eq. 1 for i<l^{V}, (l^{V}=l−w−1, i.e. the effective length for start position of kmer count vectors.) and Vᵢ=[0, . . . , 0], otherwise.

$${\tilde{V}}_{J} = \frac{{V}_{i}}{‖ {V}_{i} ‖}$$

for i=jd, as Vᵢ s are computed for adjacent windows, separated by

$$\begin{matrix}d . S [ m ] = \frac{1}{\vert γ \vert} {Σ}_{( {i}^{1} {i}^{2} ) ∈ {γ}_{m}} {S}_{{i}^{1} {i}^{2}} \\ = \frac{1}{\vert γ \vert} {\sum }_{( {j}^{1} , {j}^{2} ) ∈ {Ω}_{m}} {\tilde{V}}_{{j}^{1}}^{1} \cdot {\tilde{V}}_{{j}^{2}}^{2} \\ = \frac{1}{\vert γ \vert} {\sum }_{( {j}^{1} , {j}^{2} ) ∈ {Ω}_{m}} {\sum }_{h = 0}^{q = {\vert Σ \vert}^{k}} {\tilde{V}}_{{j}^{1} , h}^{1} {\tilde{V}}_{{j}^{2} , h}^{2} \\ = \frac{1}{\vert γ \vert} {{\sum }_{h = 0}}^{q} {\sum }_{( {j}^{1} , {j}^{2} ) ∈ {Ω}_{m}} {\tilde{V}}_{{j}^{1} , h}^{1} {\tilde{V}}_{{j}^{2} , h}^{2} \\ = \frac{1}{\vert γ \vert} {{\sum }_{h = 0}}^{q} ( {\tilde{V}}_{. , h}^{1} * {\tilde{V}}_{. , h}^{2} ) [ m ] \\ = \frac{1}{\vert γ \vert} {{\sum }_{h = 0}}^{q} ( conj ( {\tilde{V}}_{- . , h}^{1} ) * {\tilde{V}}_{. , h}^{2} ) [ m ] \\ = \frac{1}{\vert γ \vert} {IDFT ( {{\sum }_{h = 0}}^{q} conj ( {DFT ( {\tilde{V}}_{. , h}^{1} )}_{{l}_{DFT}} ) {DFT ( {\tilde{V}}_{. , h}^{2} )}_{{l}_{DFT}} )}_{{l}_{DFT}} [ m ]\end{matrix}$$

$$where$$

$${Ω}_{m} = \{ ( ⌊ \frac{m}{d} ⌋ , 0 ) , ( ⌊ \frac{m}{d} ⌋ + 1 , 1 ) , … , ( ⌊ \frac{m}{d} ⌋ + ⌊ \frac{{l}_{read}^{V}}{d} ⌋ , ⌊ \frac{{l}_{read}^{V}}{d} ⌋ ) \} ,$$

*: cross-correlation operation

*: convolution operation.

DFT( )_{N}: District Fourier Transform of size N

IDFT( )_{N}: Inverse District Fourier Transform of size N

$${l}_{DFT} \geq ⌊ \frac{{l}_{read}^{V}}{d} ⌋ + ⌊ \frac{{l}_{reference}^{V}}{d} ⌋ - 1$$

DFT might be computed efficiently using Fast Fourier Transform (FFT) algorithm. For larger lₘₐₓ compared to practical DFT sizes (N), overlap-add or overlap-save techniques might be used.

In the presence of insertions and deletions, aligned sequences at offset m have their error-free segments shifted with different offsets (\tilde{m}) depending on the number of preceding inserted and deleted bases (\tilde{m}=m−num_insertions+num_deletions). That is the reason, instead of observing exact sequence matches of nucleotide bases at fixed offset m, the content of short k-mers in a window starting at each base position is compared as a measure of local similarity.

In certain embodiments, the read sequence may be aligned to a segment or region of the reference sequence if the similarity score is above a threshold. In certain embodiments, the threshold may be a value that is at least 1.5 times the standard deviation (SD) or median absolute deviation (MAD) higher than the mean or median value, such as 2 times or 3 times the SD or MAD. In certain embodiments, the threshold may be calculated using the formula mean(S)+f×std(S) or median(S)+f×mad (S), in cases where many similarity scores (S) have been computed for the read sequence and different segments of the reference sequence. In some instances f=1, 2, or 3. FIG. 5 depicts alignment of a read sequence to a reference sequence. In FIG. 5, the similarity score between a read sequence and a segment of the reference sequence which is above a threshold is visible as a peak, indicating that the read sequence maps to the segment of the reference sequence. In certain embodiments, the read sequence may not be aligned to a segment or region of the reference sequence when the similarity score is below a threshold. In such an embodiment, the alignment method may include conducting the steps (a)-(f) for a different segment of the reference sequence.

In certain embodiments, the method is performed iteratively until the entire sequence of the read sequence has been compared to a segment of the reference sequence. In certain embodiments, the method is performed iteratively until the entire sequence of the read sequence has been compared to the entire reference sequence (e.g., when the reference sequence is another read sequence and the read sequences are being compared to identify overlapping read sequences).

In certain embodiments, the read sequence is divided into shorter sequences and the method is performed on the shorter sequences. In certain embodiments, read sequences of length 7000 bases or more may be split up into 2 or more equally sized, when possible, subsequences, or fragments. In other embodiments, read sequences of length 5000 bases or above, 6000 bases or above, 8000 bases or above, or 10,000 bases or above may be split into subsequences. In certain embodiments, the methods described herein may be performed using a read sequence that has been divided into shorter sequences of about 1000-7000 bases, such as, 1000-2000 bases, 1000-3000 bases, 1000-4000 bases, 1000-5000 bases, or 1000-6000 bases. In certain cases, a read sequence suspected of including insertions and/or deletions may be divided into shorter sequences.

In other embodiments, read sequences with lengths l>2000, 3000, 4000, 5000, 6000, 7000, and 8000 bases may be split into subsequences. This is done because as the read sequence length increases, absolute (#insertions−#deletions) of the read sequence with respect to the reference sequence may become comparable to window length w towards the end of the read, and cross correlation between the read query window and the reference sequence window become less effective and noisy. In such embodiments where the read sequence is split into subsequences, each subsequence of the original read sequence is separately aligned to the reference sequence, repeating the steps of creating windows, counting k-mers, computing k-mer count similarity values, computing a similarity score, and aligning the subsequence to a reference sequence segment for each of the subsequences.

In further embodiments, the method further comprises merging the read subsequences back together to ascertain one alignment for the read sequence to the reference sequence. In certain embodiments, each read subsequence is aligned to a region of the reference sequence at a peak position. Compatible peak positions from read subsequences are merged back together. In some instances, the exact start positions are computed for top selected peaks using banded dynamic programming (BDP) between read sequence and selected reference sequence segment in the range ([p−o, p+l+o]) where p is the detected peak position, l is the read length, and o is a margin considered due to peak position detection inaccuracy. In certain embodiments, o=2×d. In some cases, the default scoring settings for BDP is: match: +5, mismatch: −4, open gap: −10, extend gap: −1. As mentioned for certain embodiments, top peaks are detected based on average and standard deviation of S across the reference sequence. In certain embodiments, other positions within g×std(S) of maximum peak value are also considered in BDP stage, up to a maximum number of Nₘₐₓ positions, where g>0 and Nₘₐₓ can range from 1-1000 peaks. If no significant peak is detected for a read, top Nₘₐₓ are selected for the merging of read subsequences in the BDP stage. The analytical steps in the disclosed methods may be implemented in any suitable programming language, such as C, C++, Java, C#, Fortran, Pascal, or the like.

The advantage of calculating a score based on counts of k-mers within localized windows is two-fold. First, the algorithm is robust to indels since it does not seek exact matches between long stretches of the sequences. Instead, counts of overlapping k-mers are tallied, allowing for insertions and deletions to shift read sequence segments from the corresponding reference sequence segments without losing the similarity information between those segments. Second, relatively long read queries can be accurately aligned by combining the method of computing an alignment similarity score and banded dynamic programming techniques to stitch together read subsequences. Such a robust and accurate alignment method using cross-correlation provides a valuable alternative to seed-and-extend algorithms, which attempt to find clusters of exact matches between query and reference, for accurately and efficiently mapping sequences to a larger database.

In certain embodiments, the methods are computer implemented methods. In certain embodiments, the algorithm and/or results (e.g., optimal alignments between read and reference sequences) are stored on computer-readable medium, and/or displayed on a screen or on a paper print-out. In certain embodiments, the results are further analyzed, e.g., to identify genetic variants, to identify one or more origins of the sequence information, to identify genomic regions conserved between individuals or species, or to determine relatedness between two individual.

The functional aspects of the disclosed methods that are implemented on a computer may be implemented or accomplished using any appropriate implementation environment or programming language, such as C, C++, Cobol, Pascal, Java, JavaScript, HTML, XML, dHTML, assembly or machine code programming, RTL, etc.

In certain embodiments, the computer-readable media may comprise any combination of a hard drive, auxiliary memory, external memory, server, database, protable memory device (CD-R, DVD, ZIP disk, flash memory cards, etc.), and the like.

Read and Reference Sequences

Any high-throughput technique for sequencing can be used in the practice of the methods disclosed herein. DNA sequencing techniques include dideoxy sequencing reactions (Sanger method) using labeled terminators or primers and gel separation in slab or capillary, sequencing by synthesis using reversibly terminated labeled nucleotides, pyrosequencing, 454 sequencing, sequencing by synthesis using allele specific hybridization to a library of labeled clones followed by ligation, real time monitoring of the incorporation of labeled nucleotides during a polymerization step, polony sequencing, SOLID sequencing, and the like. These sequencing approaches can thus be used to sequence target nucleic acids of interest and obtain query read sequences. Reference sequences may be likewise sequenced (e.g., the reference sequence may be a read sequence to be aligned against other read sequence(s) obtained from the sequencing of a nucleic acid sample), or may be obtained through public databases, such as a national DNA database, and may take the form of one or multiple sequences, like a genome. In some embodiments, the reference sequence is a sequence for the target nucleic acid in a reference database, such as GenBank®.

The read sequence may be obtained for nucleic acid of any subject. The subject may be an organism, such as, a single celled organism (e.g., bacteria, archaea, protozoa, unicellular algae and unicellular fungi) or a multicellular organism (e.g., sponges, cnidarians, flatworms, arthropods, echinoderms, chordates, vertebrates, ferns, angiosperms, and gymnosperms). In certain cases, the read sequence may be obtained from an infectious organism, a pathogen, such as, Neisseria, HIV, E. coli, Salmonella, and the like.

The read and reference sequences may be obtained from the same species, subspecies, strain, or most closely related organisms. For example, read sequences from a human may be compared to a reference sequence from another human, such as a version of the human genome. In certain embodiments, the reference sequence(s) may be from an organism that is evolutionarily or biologically closely related to the organism from which the read sequence was obtained so that high alignment accuracy can be achieved.

In certain embodiments, the disclosed methods can be applied in finding read overlaps (i.e. pairwise alignment of read sequences). In such cases, the reference sequence would be another read sequence.

As discussed herein, the read sequence is a sequence of contiguous nucleotides determined from a single fragment of a sample nucleic acid by a sequencing instrument. In certain embodiments, the read sequence is not pre-assembled by assembling separate read sequences having overlapping regions, at which the nucleotide sequence is highly similar or identical. In other words, the read sequence may be the sequence of contiguous nucleotides obtained from sequencing of a single nucleic acid fragment generated from the genome of an organism. The read sequence length can vary, ranging from 1-20,000 bases, 1-15,000 bases, 50-15,000 bases, 100-15,000 bases, 100-10,000 bases, 100-9000 bases, 100-8000 bases, 100-7000 bases, 100-6000 bases, 100-5000 bases, 100-2500 bases, 500-10,000 bases, 500-7500 bases, 500-5000 bases, and 500-2500 bases in length.

### Computational Software & System

As noted herein, the methods provided in this application can be implemented in hardware and/or software. In some embodiments, different aspects of the methods can be implemented in either client-side logic or server-side logic. In certain cases, components used for implementing the disclosed methods may be embodied in a fixed media program component containing logic instructions and/or data that when loaded into an appropriately configured computing device causes that device to perform the method steps. A fixed media containing logic instructions may be delivered to a viewer's computer or a fixed media containing logic instructions may reside on a remote server that a viewer accesses through a communication medium in order to download a program component.

In certain aspects, the methods provided herein are computer-implemented methods, wherein at least one or more steps of the method are carried out by a computer program. A computer system for implementing the present computer-implemented method may include any arrangement of components as is commonly used in the art. In specific embodiments, the disclosed methods may be embodied in whole or in part as software recorded on fixed media. The computer system may be any electronic device including a memory, a processor, input and output devices (I/O), a data repository, a network interface, storage devices, power sources, and the like. The memory or storage device may be configured to store instructions that enable the processor to implement the present computer-implemented method by processing and executing the instructions stored in the memory or storage device. The computer may also include a network interface for wired and/or wireless communication.

The processor controls operation of the computer and may read information from the memory and/or a data repository and execute the instructions accordingly to implement the aforementioned embodiments. The term “processor” is intended to include one processor, multiple processors, or one or more processors with multiple cores.

The I/O may include any type of input devices such as a keyboard, a mouse, a microphone, etc., and any type of output devices such as a monitor and a printer, for example. In an embodiment where the computer comprises a server, the output devices may be coupled to a local client computer.

The memory may comprise any type of non-transitory, static or dynamic memory, including flash memory, DRAM, SRAM, and the like. The memory may store programs and data, which may be used in the process of sequence alignment as described herein.

The data repository may store several databases including one or more databases that store read sequences, reference sequences, k-mer count vectors, and the like. In one embodiment, the data repository may reside within the computer. In another embodiment, the data repository may be connected to the computer via a network port or external drive. The data repository may comprise a separate server or any type of memory storage device (e.g., a disk-type optical or magnetic media, solid state dynamic or static memory, and the like). The data repository may optionally comprise multiple auxiliary memory devices, e.g., for separate storage of input sequences (e.g., read sequences), sequence information, calculation results, and/or other information. The computer can thereafter use that information to direct server or client logic, as understood in the art, to embody aspects of the disclosed methods.

In operation, an operator may interact with the computer via a user interface presented on a display screen to specify the read sequences and other parameters required by the various software programs. Once invoked, the programs in the memory are executed by the processor to implement the present methods.

In one embodiment of the computer-implemented method, a user may access a file on a computer system, wherein the file contains the read sequence(s) and reference sequence(s) data, as well as a user- and computer-executable method to carry out the disclosed methods. In further embodiments, the results of the process may optionally further comprise quality information, technology information (e.g., peak characteristics, expected error rates), alternate (e.g., second or third best) consensus determination, confidence metrics, and the like.

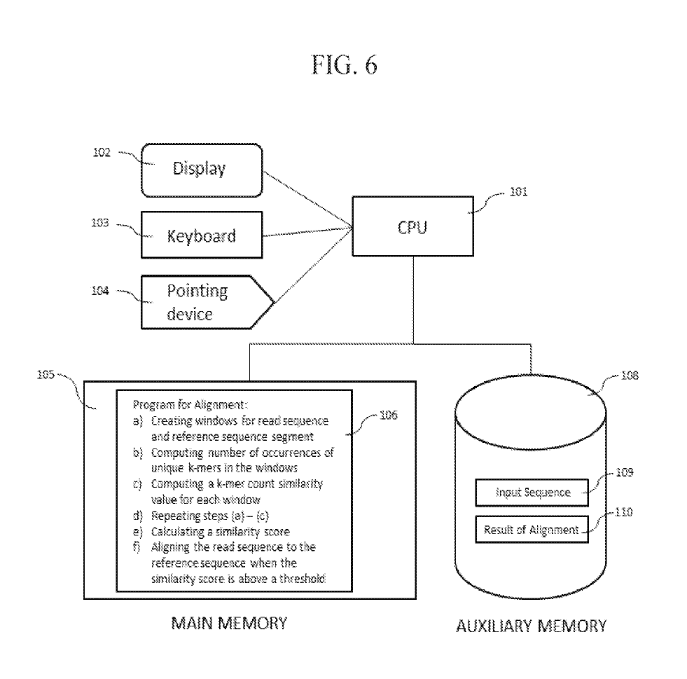

FIG. 6 illustrates one embodiment of a computer comprising memory in which instructions for carrying out the disclosed methods are stored. The computer's processor executes the stored instructions to perform alignments. This computer system includes a CPU 101 for executing instructions stored in the main memory 105, a display 102 for displaying an interface, a keyboard 103, and a pointing device 104, main memory 105 storing various programs and a storage device such as an auxiliary memory 108 that can store the input sequence 109, and results of alignment 110. The device is not limited to a personal computer, but can be any information appliance for interacting with a remote data application, and could include such devices as a digitally enabled television, cell phone, personal digital assistant, etc. Information residing in the main memory 105 and the auxiliary memory 108 may be used to program such a system and may represent a disk-dynamic or static memory, etc. In specific embodiments, the disclosed methods may be embodied in whole or in part as software recorded on this fixed media. The various programs stored on the main memory can include a program 106 to align a read sequence to a reference sequence using the methods disclosed herein. The lines connecting CPU 101, main memory 105, and auxiliary memory 108 may represent any type of communication connection. For example, auxiliary memory 108 may reside within the device or may be connected to the device via, e.g., a network port or external drive. Auxiliary memory 108 may reside on any type of memory storage device (e.g., a server or media such as a CD or floppy drive), and may optionally comprise multiple auxiliary memory devices, e.g., for separate storage of input sequences, results of alignment, results of result interpretation, and/or other information.

The output of the alignment analysis may be provided in any convenient form. In some embodiments, the output is provided on a user interface, a print out, in a database, etc. and the output may be in the form of a table, graph, raster plot, heat map, and the like. In certain embodiments, the output of the implementation of the alignment method may include a list of alignments for each read sequence to a position in a reference sequence, in multiple reference sequences, or another read sequence. In certain embodiments, the results of the process may optionally further comprise technology information (e.g., peak characteristics, expected error rates), alternate (e.g., second or third best) alignments, confidence metrics, and the like. During and after the process of aligning a read sequence to a reference sequence, the progress and/or result of this processing may be saved to the memory and the data repository and/or output through the I/O for display on a display device and/or saved to an additional storage device (e.g., CD, DVD, Blu-ray, flash memory card, etc.), transmitted or printed.

### EXAMPLES

Below are examples of specific embodiments for carrying out the present invention. The examples are offered for illustrative purposes only, and are not intended to limit the scope of the present invention in any way.

Efforts have been made to ensure accuracy with respect to numbers used (e.g., amounts, temperatures, etc.), but some experimental error and deviation should, of course, be allowed for.

### Example 1

Demonstrating the Effectiveness of the Cosine Similarity Metric Using the E. coli Genome.

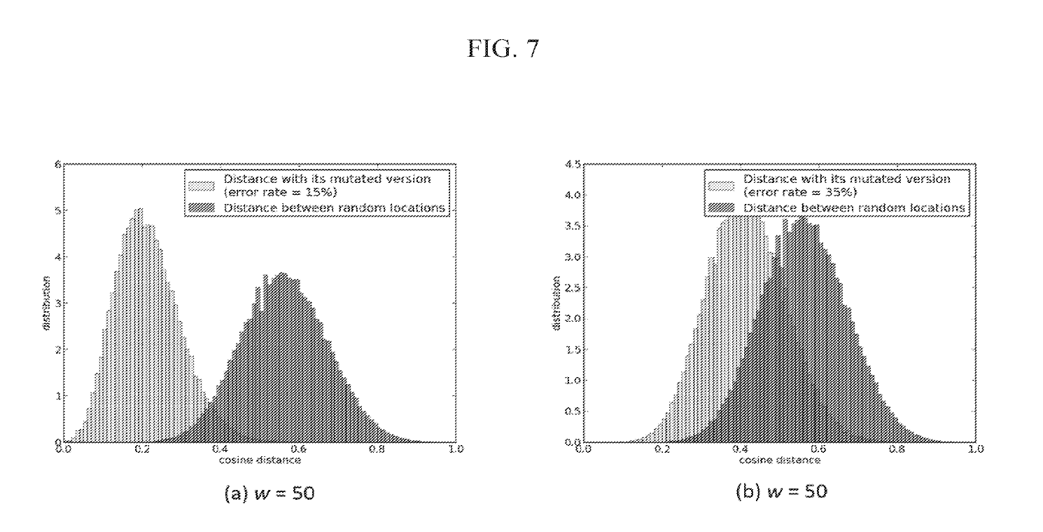

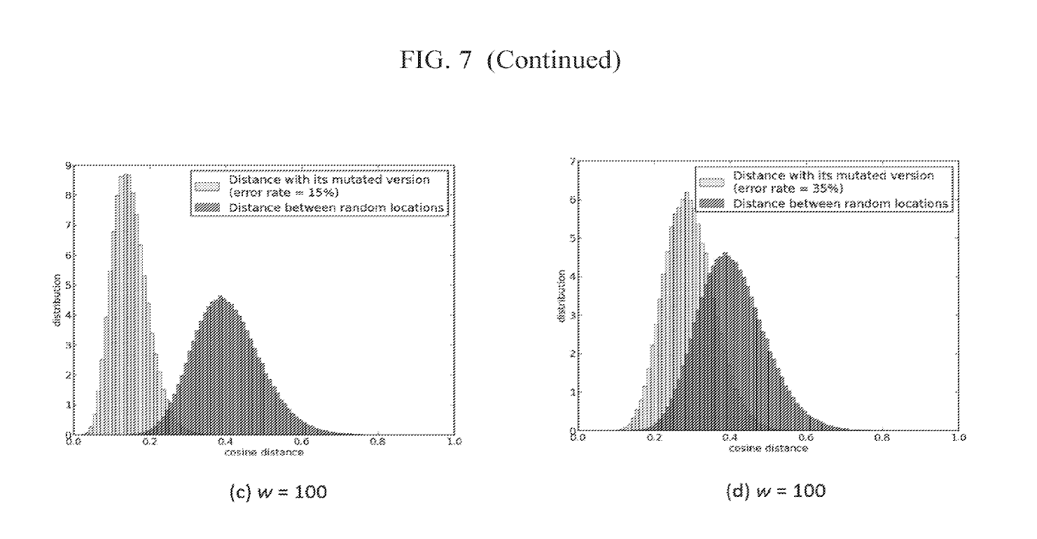

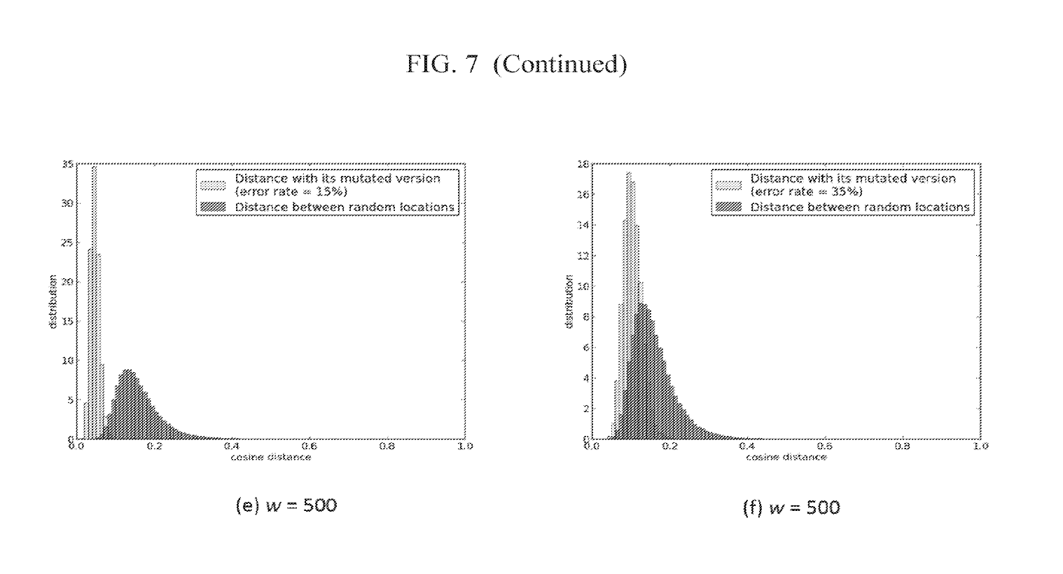

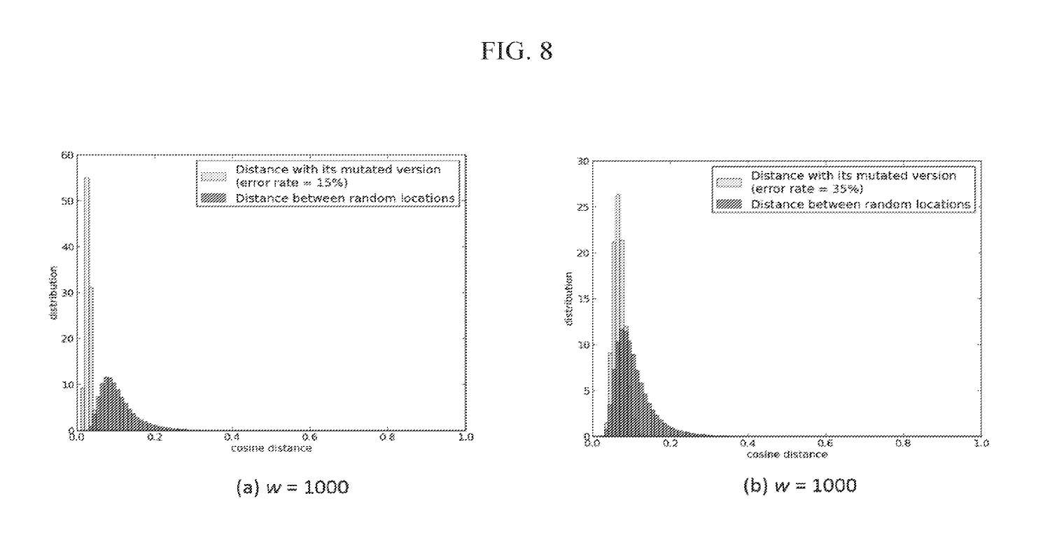

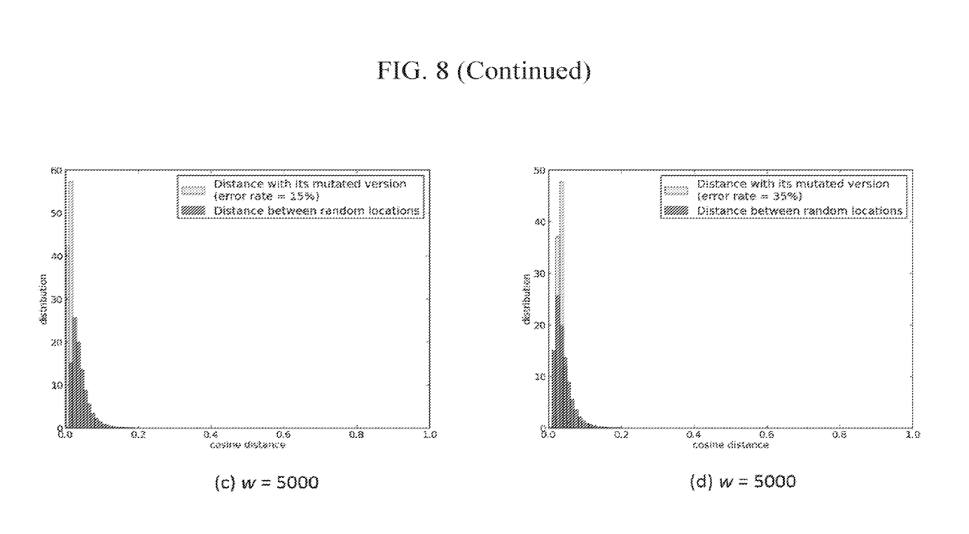

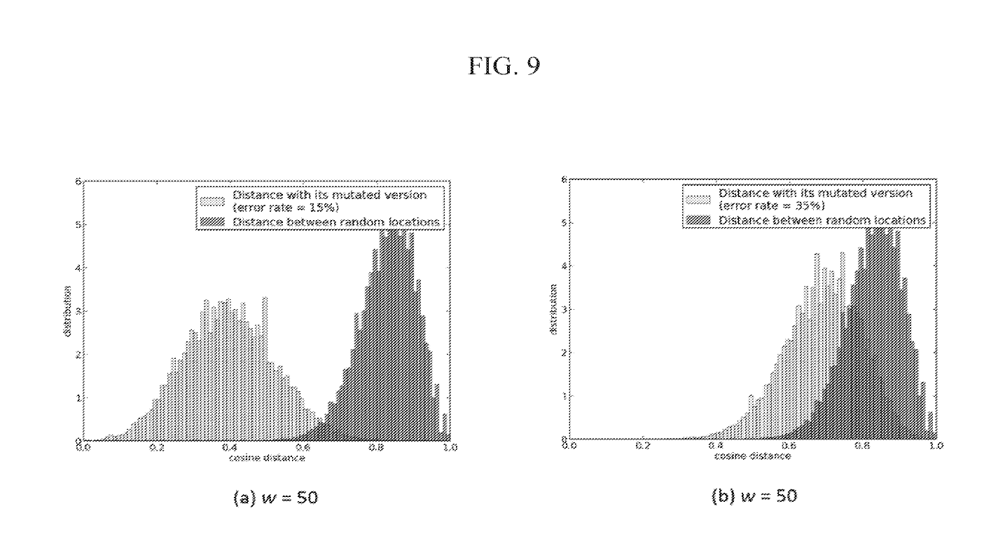

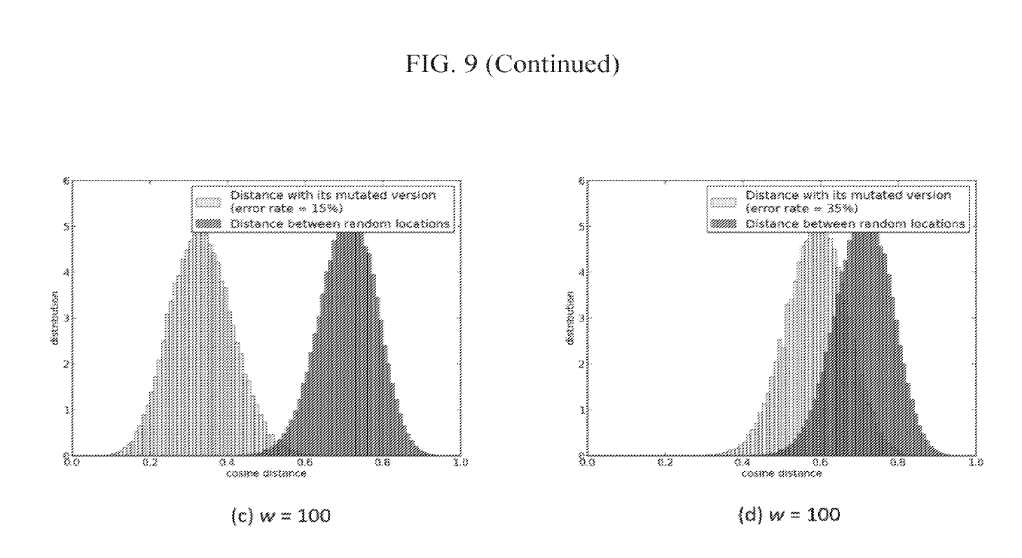

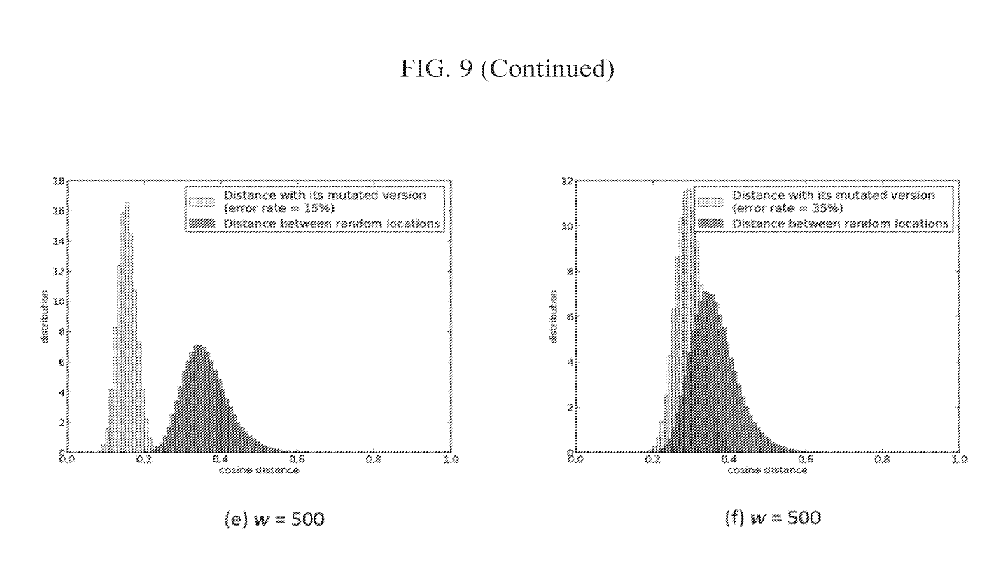

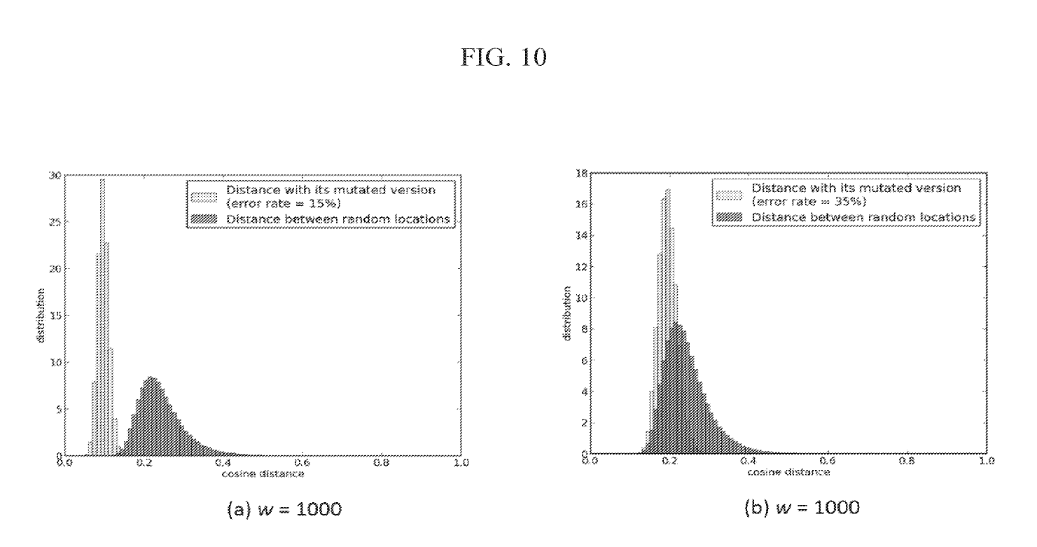

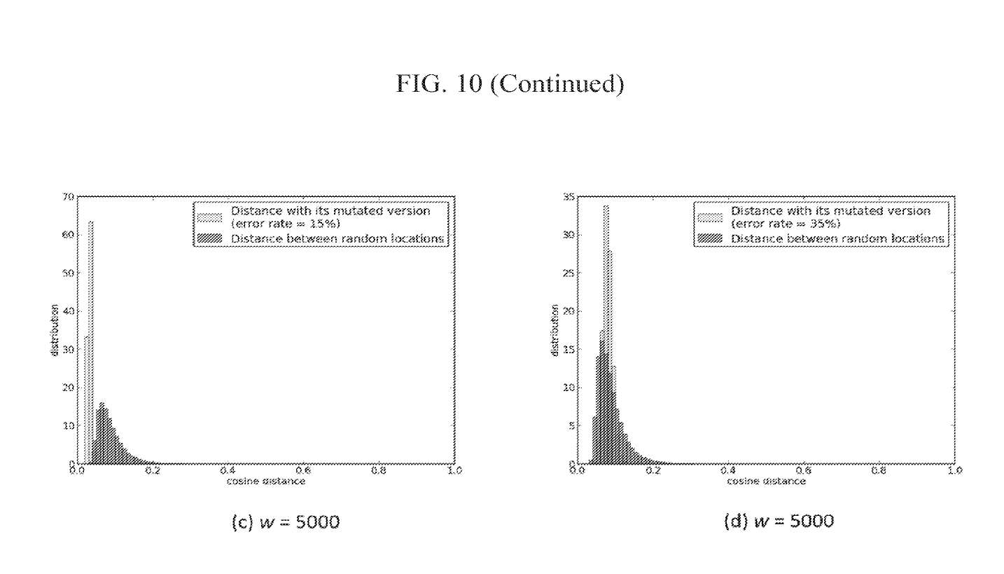

A cosine similarity is a metric used to determine the similarity between two vectors by measuring the cosine of the angle between them. To demonstrate the effectiveness of this metric, 1000 sequences of length 5000 bases each were selected from random locations in the E. coli genome. For each sequence, a cosine distance (1−cosine similarity) was computed between non-overlapping windows of different lengths w=50, 100, 500, 1000, and 5000 bases, and between each window's sequence and its 10 randomly mutated versions with average substitution rates of 15% and 35%. FIGS. 7-8 and 9-10 present the cosine distance distribution for k=3 and k=4, respectively. FIGS. 7-8 and 9-10 illustrate how the distribution of cosine distance between short k-mer count vectors at random positions are distinguishable from their mutated versions. Furthermore, as expected, the distributions overlap becomes significant for higher mutation rates, which increase the dissimilarity between a window and its mutated version. In the graphs of FIGS. 7-10, the left distribution is the “distance with its mutated version” with varying mutation rates, and the right distribution is the “distance between random locations,” with varying mutation rates.

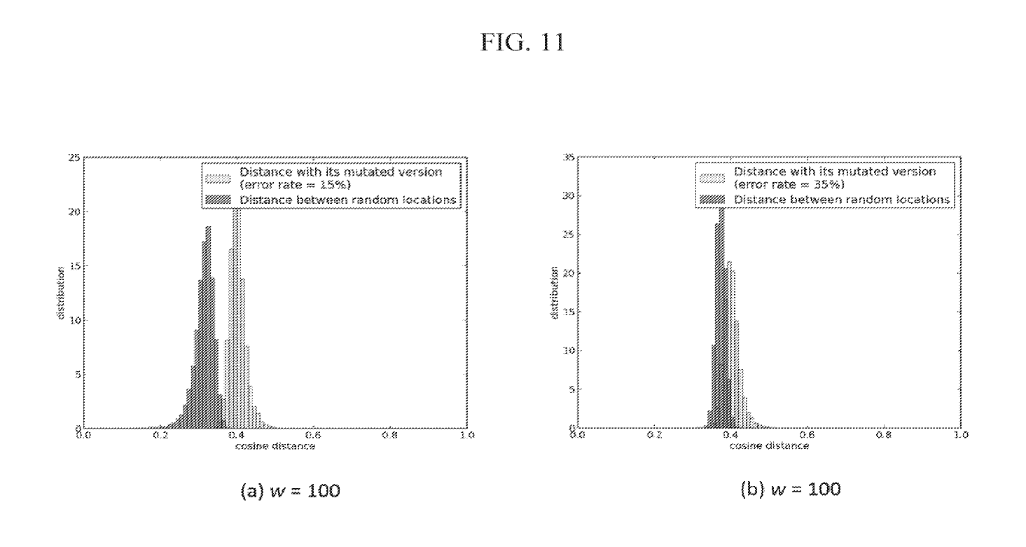

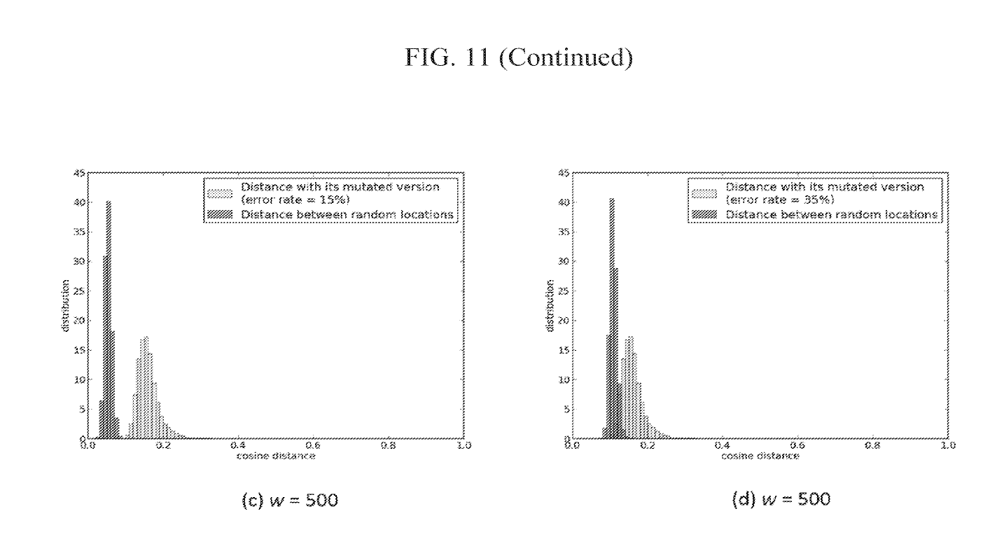

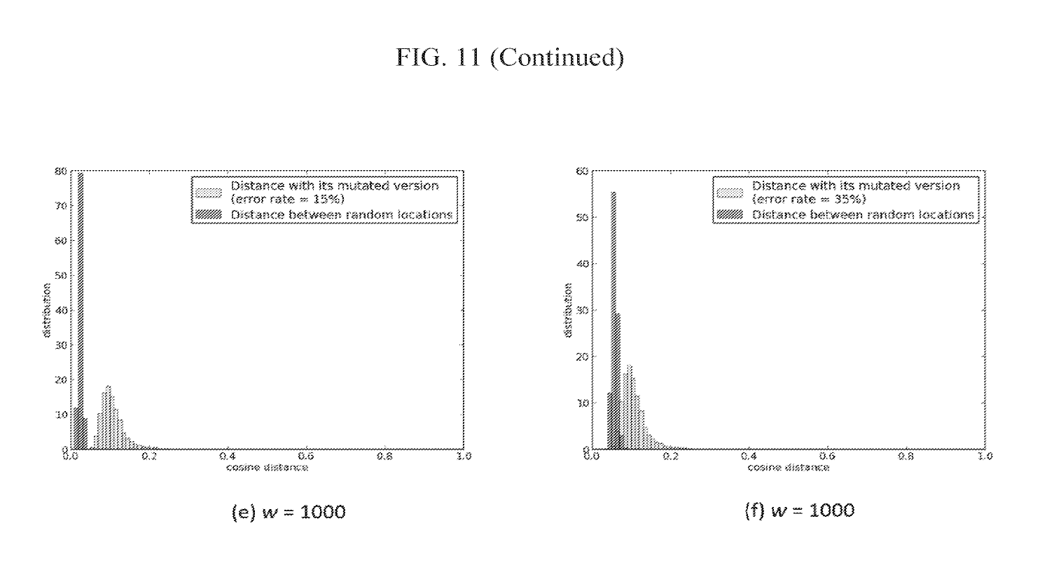

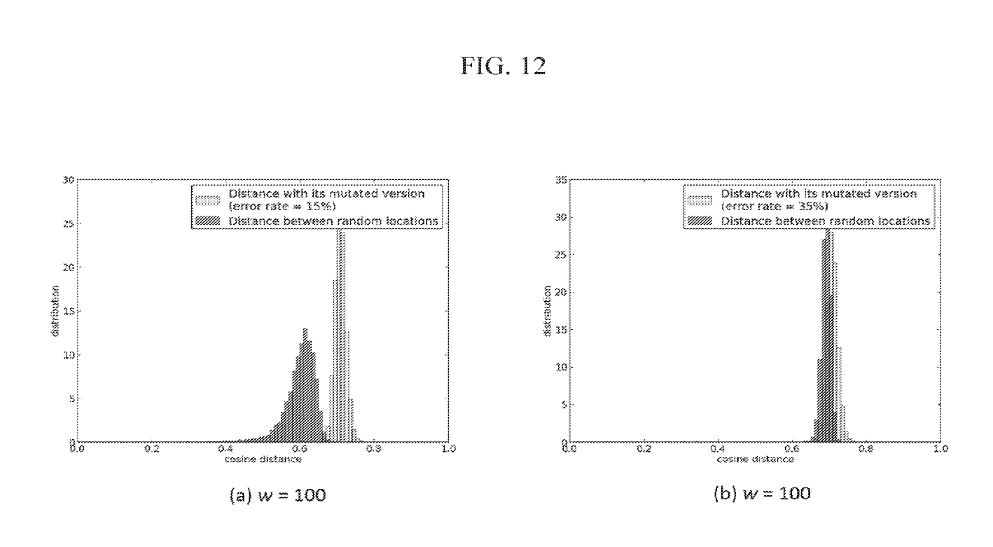

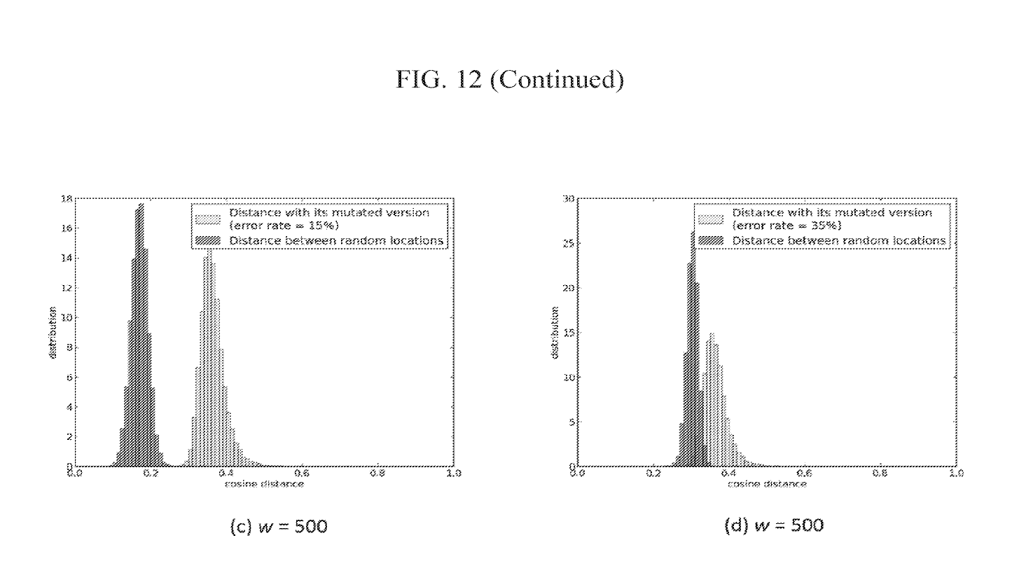

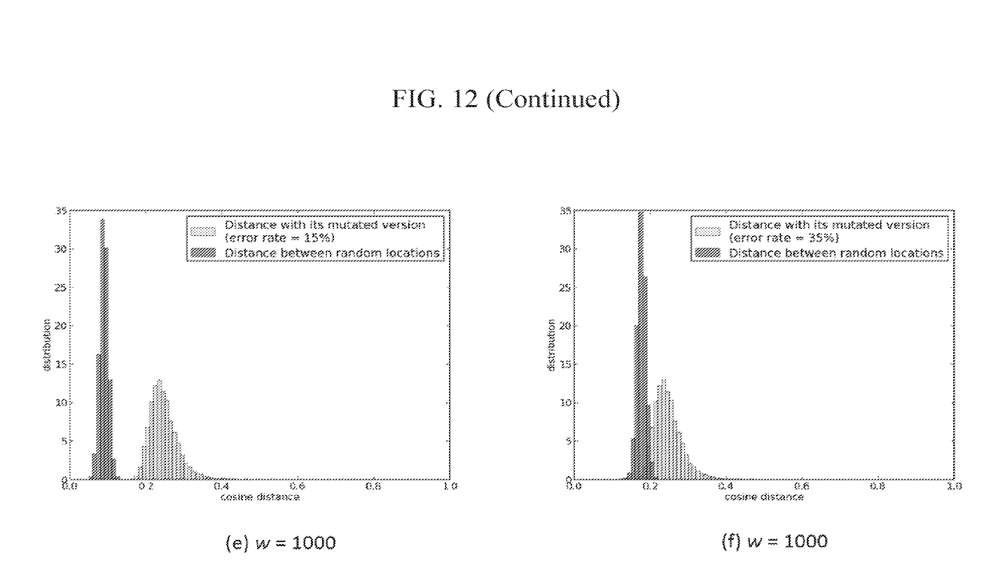

In addition to computing the cosine similarity between single windows with mutation patterns consisting solely of substitutions, multiple windows and the addition of indels was tested. Again, 1000 read sequences of length 5000 bases were selected from random locations in the E. coli genome. A similarity distance (1−S[0]) was computed between each read sequence and 10 other random non-overlapping read sequences, and between each read sequence and its 10 randomly mutated versions in the presence of error rates of 15% and 35%, using window shift size of d=10. An error pattern of 10% substitutions, 28% deletions, and 62% insertions is used. FIGS. 11 and 12 show the similarity distribution of this test for k=3 and k=4, respectively. In the graphs of FIGS. 11 and 12, the left distribution is the “distance with its mutated version” with varying error rates, and the right distribution is the “distance between random locations,” with varying error rates. Due to relatively higher indel rates, misalignment between smaller window sizes has a negative effect on similarity score. On the contrary, using a large (w>1000 bases) window size loses the locality information of k-mers at each position.

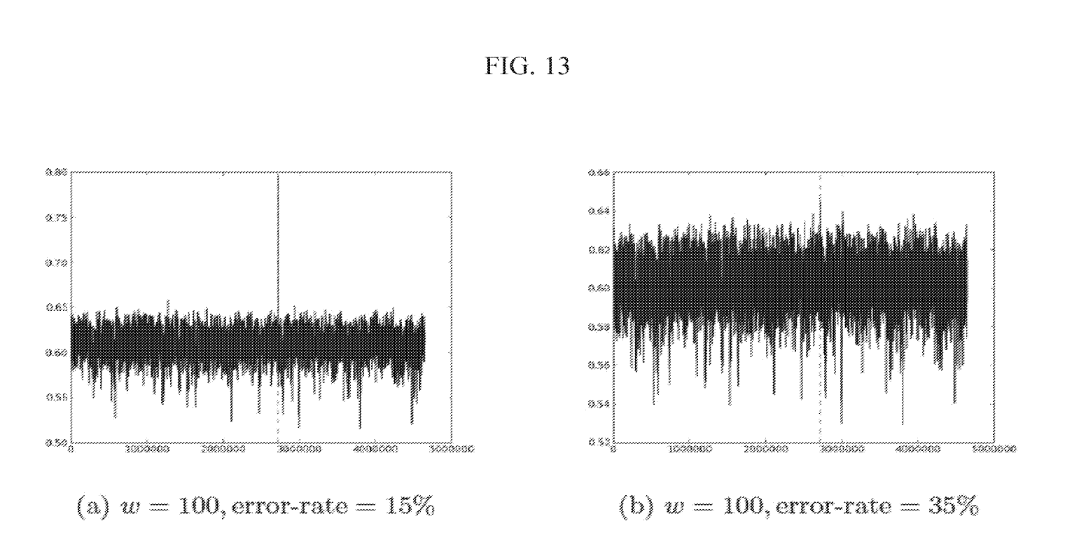

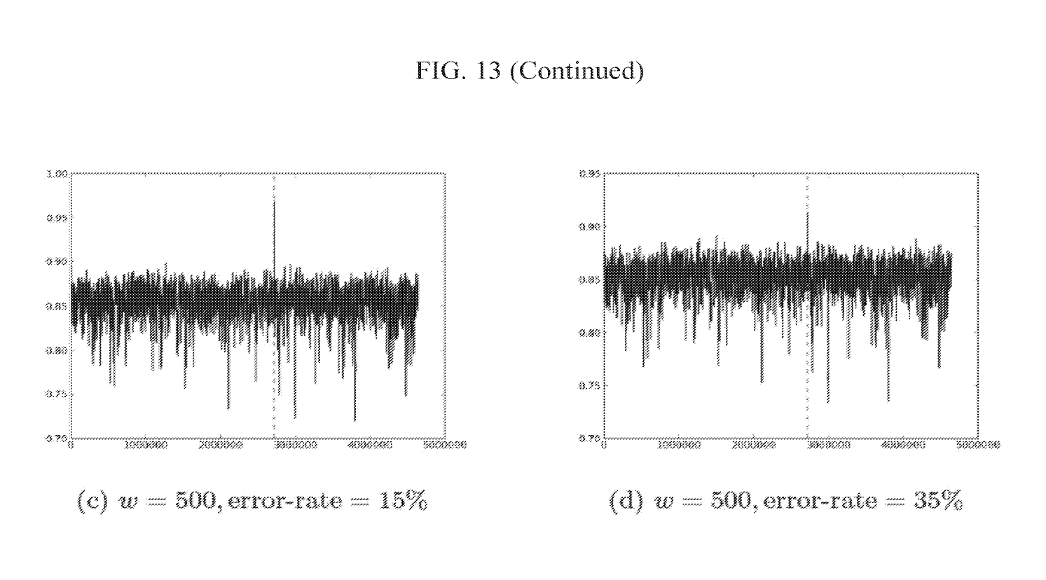

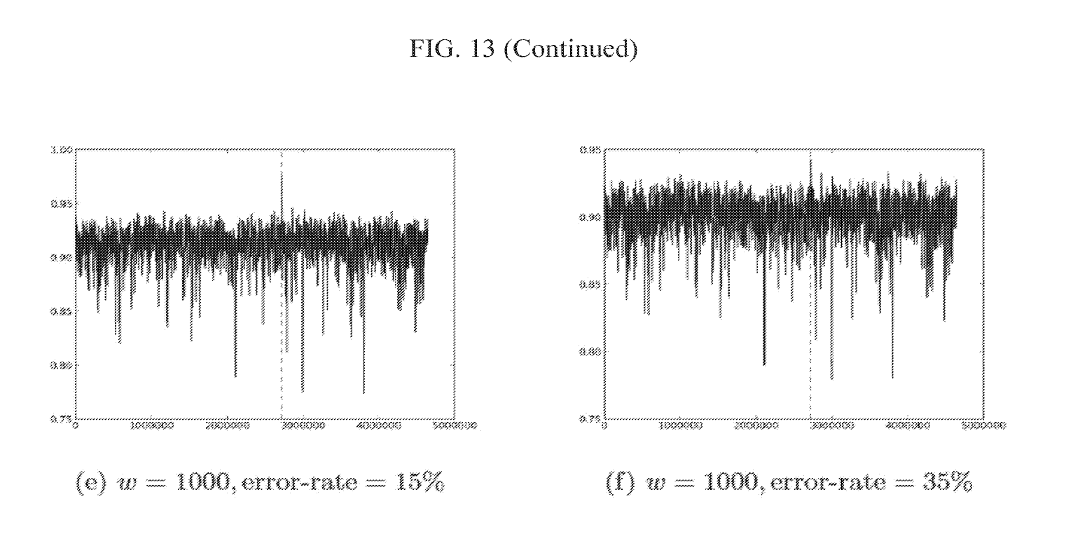

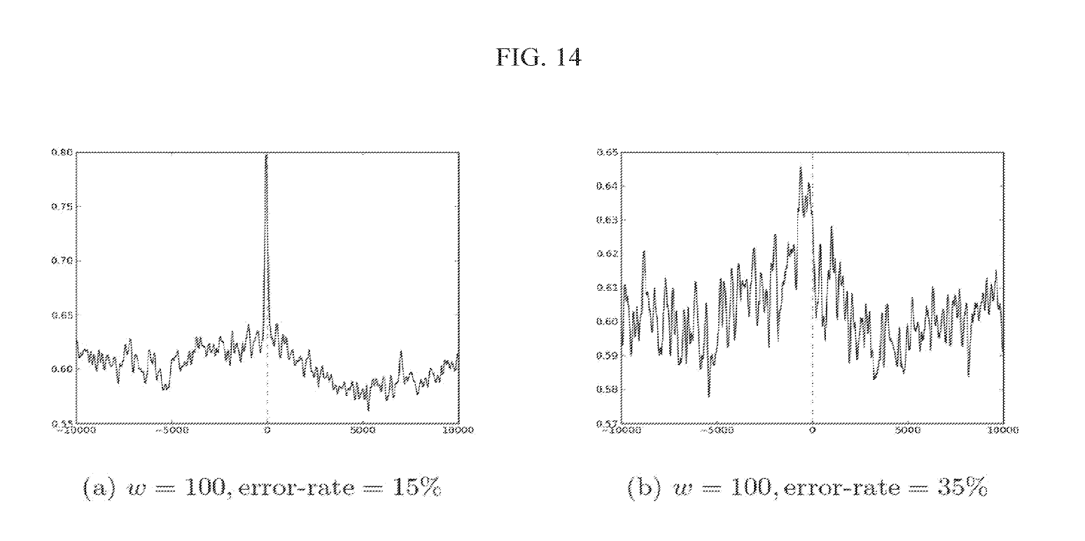

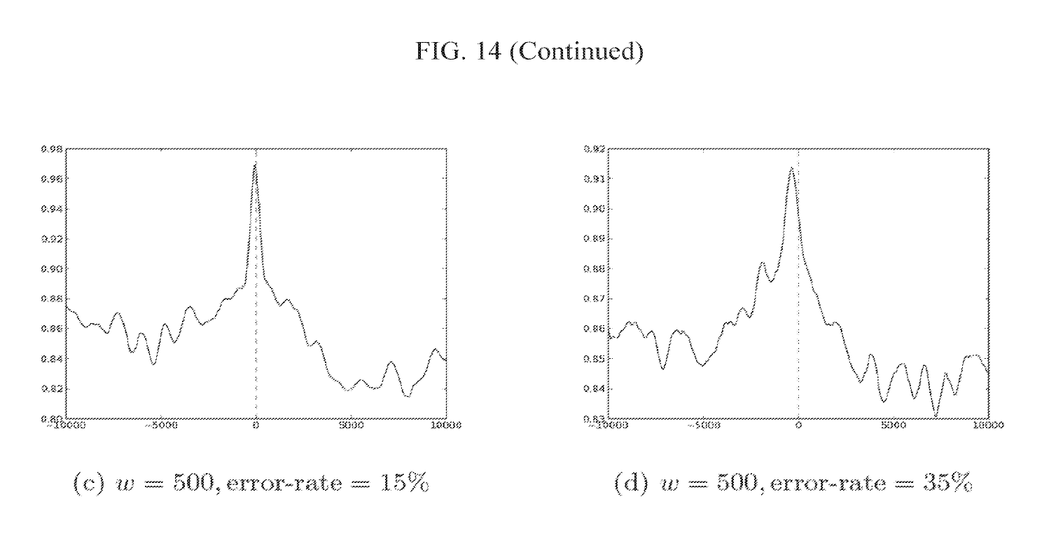

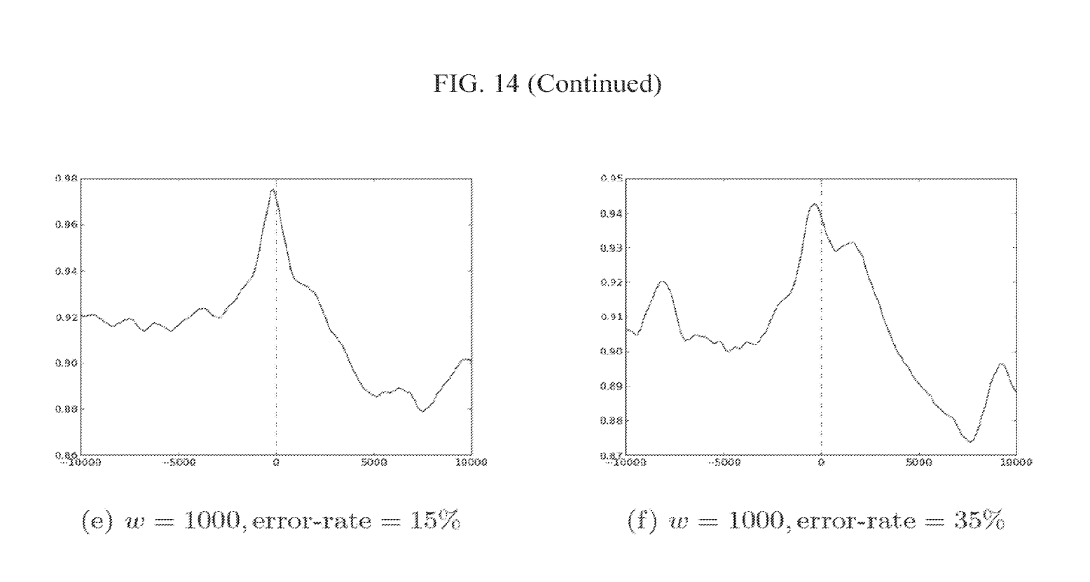

In order to find the optimal window size, a read sequence was extracted from E. coli K12 region, starting at a sampled position 2,720,230-2,725,230 and simulated with 15% and 35% error rates. As an example, FIG. 13 shows the overall similarity scores (S[m]) for a read across whole E. coli genome using k=3 and d=10 settings. High similarity scores S[m], indicated by the peaks in the graphs of FIG. 14, are detectable close to the sampled position. FIG. 14 illustrates the score around the sampled position (x-axis centered at 2,720,230), which shows the trade-off in choice of window size. For shorter window sizes (FIG. 14a, 14b, w=100 bases), the peak becomes noisy and for larger window sizes (FIG. 14e, 14f, w=1000 bases), the peak becomes wider, both reducing the accuracy in detecting the correct start position.

Taken together, the data shows that the presently disclosed algorithm can be optimized to utilize a window size between 100-1000 bases in order to accurately and efficiently identify the similarity between two sequences, even in the presence of relatively high error rates, including indels. These data show also that the cosine similarity score is an accurate metric and more efficient compared to other distance formulas since it can be implemented efficiently using Fast Fourier Transom (FFT).

An experimental code was implemented in C programming language to evaluate the algorithm on a GPU setup. The GPU setup in use was Nvidia Titan X (12G GDDR5). In general, for a sequence of length 1, computing the normalized k-mer count vectors (\tilde{V}ᵢ) takes O(l) in time and computing their Fast Fourier transform per k-mer takes O(l_{DFT} log(l_{DFT})) and in total (ql_{DFT} log(l_{DFT})). Computing the overall score S which includes multiplying the reference and query FFT vectors and computing its inverse FFT is in O(l_{DFT}(q+log(l_{DFT}))) and the computation time for banded dynamic programming (BDP) is in O(l_{lead}band_len) (where band_len is O(l_{lead}abs(insertion_rate−deletion_rate)). The FFT and IFFT step might be computed efficiently by splitting a large reference sequence to short segments of optimal transform size N and using an overlap-save (or overlap-add) technique (Oppenheim et al., 2009). The FFT and BDP operations are implemented using NVIDIA cuFFT and NVBIO libraries.

### Example 2

Accuracy and Performance Analysis Using E. coli Genome

Accuracy and performance of this method was evaluated using 20× simulated read datasets from E. coli genome with average length of 5 kbps and 10 kbps and different sequence accuracies of 85%, 75%, 65% and 55%. Read sequences were simulated using PBSIM (Ono et al., 2013) with option (--data-type CLR --depth 20 --model_qc model_qc_clr --accuracy-min 0.5 --length-mean [5000110000]--length-sd 2000 --accuracy-mean [0.85|0.75|0.65|0.55]--accuracy-sd 0.02).

The performance is reported for k=3, 4 with default settings of (w=500, Lₜ=7500, f=2, g=1, max-num-top-peaks=10, max-fft-block-size=32768 in Tables 1 and 2 for datasets of average sequence length of 5 kbps and 10 kbps, respectively. k=4 has almost perfect accuracy even in case of ˜45% error rate. As expected from Table 2, longer reads resulted in overall higher alignment rate specially in locating the reads that cover long repeat regions. Reads are tagged as skipped if l_{read}<w which occurs rarely given the distribution of sequence length in simulated datasets. Table 3 also reports the performance in aligning ˜45,000 simulated reads (avg. 5 Kbps long) to human chr1.

TABLE 1
Alignment accuracy on 20X simulated datasets from E. coli genome
with different error rates. Average read sequence length is 5 kbps.

| | | | |
|---|---|---|---|
| | correctly mapped | incorrectly mapped | skipped |
| read accuracy | reads | reads | reads |

k = 3, d = 10

| | | | |
|---|---|---|---|
| 85% | 18458 (1.000) | 0 (0.000) | 0 (0.000) |
| 75% | 18653 (1.000) | 1 (0.000) | 1 (0.000) |
| 65% | 18613 (0.999) | 10 (0.001) | 1 (0.000) |
| 55% | 18103 (0.975) | 455 (0.025) | 0 (0.000) |

k = 4, d = 10

| | | | |
|---|---|---|---|
| 85% | 18458 (1.000) | 0 (0.000) | 0 (0.000) |
| 75% | 18653 (1.000) | 1 (0.000) | 1 (0.000) |
| 65% | 18621 (1.000) | 2 (0.000) | 1 (0.000) |
| 55% | 18530 (0.998) | 27 (0.001) | 1 (0.000) |

k = 5, d = 10

| | | | |
|---|---|---|---|
| 85% | 18458 (1.000) | 0 (0.000) | 0 (0.000) |
| 75% | 18653 (1.000) | 1 (0.000) | 1 (0.000) |
| 65% | 18622 (1.000) | 1 (0.000) | 1 (0.000) |
| 55% | 18555 (1.000) | 2 (0.000) | 1 (0.000) |

TABLE 2
Alignment accuracy on 20X simulated datasets from E. coli genome
with different error rates. Average read sequence length is 10 kbps.

| | | | |
|---|---|---|---|
| | correctly mapped | incorrectly mapped | skipped |
| read accuracy | reads | reads | reads |

k = 3, d = 10

| | | | |
|---|---|---|---|
| 85% | 9274 (1.000) | 0 (0.000) | 0 (0.000) |
| 75% | 9255 (1.000) | 0 (0.000) | 2 (0.000) |
| 65% | 9280 (1.000) | 0 (0.000) | 0 (0.000) |
| 55% | 9232 (0.994) | 52 (0.006) | 0 (0.000) |

k = 4, d = 10

| | | | |
|---|---|---|---|
| 85% | 9274 (1.000) | 0 (0.000) | 0 (0.000) |
| 75% | 9255 (1.000) | 0 (0.000) | 2 (0.000) |
| 65% | 9280 (1.000) | 0 (0.000) | 0 (0.000) |
| 55% | 9283 (1.000) | 1 (0.000) | 0 (0.000) |

k = 5, d = 10

| | | | |
|---|---|---|---|
| 85% | 9274 (1.000) | 0 (0.000) | 0 (0.000) |
| 75% | 9255 (1.000) | 0 (0.000) | 2 (0.000) |
| 65% | 9280 (1.000) | 0 (0.000) | 0 (0.000) |
| 55% | 9284 (1.000) | 0 (0.000) | 0 (0.000) |

TABLE 3
Alignment accuracy on 1X simulated datasets from
human chromosome 1 with different error rates.
Average read sequence length is 5 kbps.

| | | | |
|---|---|---|---|
| | correctly mapped | incorrectly mapped | skipped |
| read accuracy | reads | reads | reads |

k = 4, d = 100

| | | | |
|---|---|---|---|
| 85% | 44701 (0.998) | 68 (0.002) | 0 (0.000) |
| 75% | 44769 (0.998) | 111 (0.002) | 0 (0.000) |
| 65% | 44744 (0.997) | 135 (0.003) | 0 (0.000) |
| 55% | 44152 (0.982) | 811 (0.018) | 0 (0.000) |

TGS reads reach tens of kpbs and they mostly have accuracy of >70%. However achieving high sensitivity with shorter segments (multi-kbps long) becomes more important in pairwise alignment of raw reads for applications such as assembly, where reads are partially overlapped and the error rate is 2× that of the raw read. There are hybrid methods developed to correct TGS reads using short second generation sequencing (SGS) sequences. But these methods require multiple library preparation and sequencing runs, also they are prone to complexities in resolving short read alignments to noisy long reads originated from repeat regions. As explained, the methods disclosed herein may be used to perform pairwise alignment of read sequences to identify overlapping read sequences.

What is claimed is:

1. A method for aligning a read sequence to a reference sequence segment, the method comprising:
a. creating a window for the read sequence and a window for the reference sequence segment, wherein the windows are of the same length;
b. computing the numbers of occurrences of unique k-mers within each window,
c. computing a k-mer count similarity value based on the numbers of occurrences of the unique k-mers within each window;
d. performing steps (a)-(c) iteratively for a plurality of windows across the read sequence and a plurality of windows across the reference sequence segment, thereby computing a plurality of k-mer count similarity values,
wherein the beginning of each subsequent window in each of the read sequence and of the reference sequence segment is offset from the beginning of the previous window in the respective sequence by a distance d;
e. calculating a similarity score by averaging the plurality of k-mer count similarity values; and
f. aligning the read sequence to the reference sequence segment when the similarity score is above a threshold,
wherein the windows created in the first performance of step (a) are positioned at the start of each sequence.

2. The method according to claim 1, wherein steps (a)-(f) are repeated for the read sequence and a different segment of the reference sequence.

3. The method according to any one of claim 1 or 2, wherein the reference sequence segment is a region of a reference sequence obtained from a genome database.

4. The method according to any one of claim 1 or 2, wherein the reference sequence is a read sequence.

5. The method according to claim 4, wherein the reference sequence that is a read sequence is obtained from sequencing the same sample from which the sequence of the read sequence in claim 1 is obtained.

6. The method according to any one of claims 1-5, wherein the length of each of the windows is at least 50 bases.

7. The method according to any one of claims 1-5, wherein the length of each of the windows can be any whole number value ranging from 1-10,000 bases, wherein the length is held constant.

8. The method according to any one of claims 1-7, wherein the distance d is at least 10 bases long.

9. The method according to any one of claims 1-7, wherein the distance d can range from 1-500 bases in length, wherein d is held constant.

10. The method according to any one of claims 1-9, wherein the k-mer is 2-10 bases in length.

11. The method according to claim 10, wherein the k-mer is 3 bases in length.

12. The method according to claim 10, wherein the k-mer is 4 bases in length.

13. An executable software product stored on a computer-readable medium containing program instructions for a method for aligning a read sequence to a reference sequence segment, the method comprising:
a. creating a window for the read sequence and a window for the reference sequence segment, wherein the windows are of the same length;
b. computing the numbers of occurrences of unique k-mers within each window,
c. computing a k-mer count similarity value based on the numbers of occurrences of the unique k-mers within each window;
d. performing steps (a)-(c) iteratively for a plurality of windows across the read sequence and a plurality of windows across the reference sequence segment, thereby computing a plurality of k-mer count similarity values,
wherein the beginning of each subsequent window in each of the read sequence and of the reference sequence segment is offset from the beginning of the previous window in the respective sequence by a distance d;
e. calculating a similarity score by averaging the plurality of k-mer count similarity values; and
f. aligning the read sequence to the reference sequence segment when the similarity score is above a threshold,
wherein the windows created in the first performance of step (a) are positioned at the start of each sequence.

14. The executable software product according to claim 13, wherein steps (a)-(f) are repeated for the read sequence and a different segment of the reference sequence.

15. The executable software product according to any one of claim 13 or 14 wherein the reference sequence segment is a region of a reference sequence obtained from a genome database.

16. The executable software product according to any one of claim 13 or 14, wherein the reference sequence is a read sequence.

17. The executable software product according to claim 16, wherein the reference sequence that is a read sequence is obtained from sequencing the same sample from which the sequence of the read sequence in claim 13 is obtained.

18. The executable software product according to any one of claims 13-17, wherein the length of each of the windows is at least 50 bases.

19. The executable software product according to any one of claims 13-17, wherein the length of each of the windows can be any whole number value ranging from 1-10,000 bases, wherein the length is held constant.

20. The executable software product according to any one of claims 13-19, wherein the distance d is at least 10 bases long.

21. The executable software product according to any one of claims 13-19, wherein the distance d can range from 1-500 bases in length, wherein d is held constant.

22. The executable software product according to any one of claims 13-21, wherein the k-mer is 2-10 bases in length.

23. The executable software product according to claim 22, wherein the k-mer is 3 bases in length.

24. The executable software product according to claim 22, wherein the k-mer is 4 bases in length.

25. A system for aligning a read sequence to a reference sequence segment, comprising:
a memory; and
a processor coupled to the memory and configured to execute instructions stored in the memory, the instructions comprising instructions for:
a. creating a window for the read sequence and a window for the reference sequence segment, wherein the windows are of the same length;
b. computing the numbers of occurrences of unique k-mers within each window,
c. computing a k-mer count similarity value based on the numbers of occurrences of the unique k-mers within each window;
d. performing steps (a)-(c) iteratively for a plurality of windows across the read sequence and a plurality of windows across the reference sequence segment, thereby computing a plurality of k-mer count similarity values,
wherein the beginning of each subsequent window in each of the read sequence and of the reference sequence segment is offset from the beginning of the previous window in the respective sequence by a distance d;
e. calculating a similarity score by averaging the plurality of k-mer count similarity values; and
f. aligning the read sequence to the reference sequence segment when the similarity score is above a threshold,
wherein the windows created in the first performance of step (a) are positioned at the start of each sequence.

26. The system according to claim 25, wherein steps (a)-(f) are repeated for the read sequence and a different segment of the reference sequence.

27. The system according to any one of claim 25 or 26, wherein the reference sequence segment is a region of a reference sequence obtained from a genome database.

28. The system according to any one of claim 25 or 26, wherein the reference sequence is a read sequence.

29. The system according to claim 28, wherein the reference sequence that is a read sequence is obtained from sequencing the same sample from which the sequence of the read sequence in claim 25 is obtained.

30. The system according to any one of claims 25-29, wherein the length of each of the windows is at least 50 bases.

31. The system according to any one of claims 25-29, wherein the length of each of the windows can be any whole number value ranging from 1-10,000 bases, wherein the length is held constant.

32. The system according to any one of claims 25-31, wherein the distance d is at least 10 bases long.

33. The system according to any one of claims 25-31, wherein the distance d can range from 1-500 bases in length, wherein d is held constant.

34. The system according to any one of claims 25-33, wherein the k-mer is 2-10 bases in length.

35. The system according to claim 34, wherein the k-mer is 3 bases in length.

36. The system according to claim 34, wherein the k-mer is 4 bases in length.

37. A storage device storing instructions that are executable to perform operations comprising:
a. creating a window for the read sequence and a window for the reference sequence segment, wherein the windows are of the same length;
b. computing the numbers of occurrences of unique k-mers within each window,
c. computing a k-mer count similarity value based on the numbers of occurrences of the unique k-mers within each window;
d. performing steps (a)-(c) iteratively for a plurality of windows across the read sequence and a plurality of windows across the reference sequence segment, thereby computing a plurality of k-mer count similarity values,
wherein the beginning of each subsequent window in each of the read sequence and of the reference sequence segment is offset from the beginning of the previous window in the respective sequence by a distance d;
e. calculating a similarity score by averaging the plurality of k-mer count similarity values; and
f. aligning the read sequence to the reference sequence segment when the similarity score is above a threshold,
wherein the windows created in the first performance of step (a) are positioned at the start of each sequence.

38. The storage device according to claim 37, wherein steps (a)-(f) are repeated for the read sequence and a different segment of the reference sequence.

39. The storage device according to any one of claim 37 or 38, wherein the reference sequence segment is a region of a reference sequence obtained from a genome database.

40. The storage device according to any one of claim 37 or 38, wherein the reference sequence is a read sequence.

41. The storage device according to claim 40, wherein the reference sequence that is a read sequence is obtained from sequencing the same sample from which the sequence of the read sequence in claim 37 is obtained.

42. The storage device according to any one of claims 37-41, wherein the length of each of the windows is at least 50 bases.

43. The storage device according to any one of claims 37-41, wherein the length of each of the windows can be any whole number value ranging from 1-10,000 bases, wherein the length is held constant.

44. The storage device according to any one of claims 37-43, wherein the distance d is at least 10 bases long.

45. The storage device according to any one of claims 37-43, wherein the distance d can range from 1-500 bases in length, wherein d is held constant.

46. The storage device according to any one of claims 37-45, wherein the k-mer is 2-10 bases in length.

47. The storage device according to claim 46, wherein the k-mer is 3 bases in length.

48. The storage device according to claim 46, wherein the k-mer is 4 bases in length.


SEQUENCE LISTING (8 sequences)

SEQ ID NO: 1 - DNA, 16 bp, Artificial Sequence
  Synthetic sequence
atgcttcgaatcgata

SEQ ID NO: 2 - DNA, 16 bp, Artificial Sequence
  Synthetic sequence
atccttcggatcgtta

SEQ ID NO: 3 - DNA, 15 bp, Artificial Sequence
  Synthetic sequence
attattagtcctagg

SEQ ID NO: 4 - DNA, 15 bp, Artificial Sequence
  Synthetic sequence
attattagatcctag

SEQ ID NO: 5 - DNA, 21 bp, Artificial Sequence
  Synthetic sequence
atgcttcgaatcgatagccta

SEQ ID NO: 6 - DNA, 21 bp, Artificial Sequence
  Synthetic sequence
atccttcggatcgttagtcta

SEQ ID NO: 7 - DNA, 31 bp, Artificial Sequence
  Synthetic sequence
  Misc_Feature (1)..(8): n is A T, C, or G
  Misc_Feature (24)..(31): n is A T, C, or G
nnnnnnnnaaaagccccggttttnnnnnnnn

SEQ ID NO: 8 - DNA, 31 bp, Artificial Sequence
  Synthetic sequence
  Misc_Feature (1)..(8): n is A, T, C, or G
  Misc_Feature (24)..(31): n is A, T, C, or G
nnnnnnnnaaaaccccggattttnnnnnnnn